(19) United States
(12) Patent Application Publication
(10) Pub. No.: US 2024/0245741 A1
(43) Pub. Date: Jul. 25, 2024

(54) EXTRACTION METHOD USING AN AQUEOUS ALKANEDIOL

(71) Applicants: Minasolve SAS, Beuvry-la-Forêt (FR); Avignon University, Avignon (FR)

(72) Inventors: Boutheina KHADHRAOUI, Mont-Saint-Guibert (BE); Markus NAHRWOLD, Mont-Saint-Guibert (BE); Farid CHEMAT, Morieres les Avignon (FR); Anne-Sylvie FABIANO TIXIER, Rochefort du Gard (FR); Karine RUIZ, Les Valayans (FR)

(21) Appl. No.: 18/690,952

(22) PCT Filed: Sep. 12, 2022
(86) PCT No.: PCT/EP2022/075241

§ 371 Date: Mar. 11, 2024

(30) Foreign Application Priority Data
Sep. 13, 2021 (EP) 21196365.7

(51) Int. Cl. A61K 36/185; A61K 8/9789; A61Q 19/00

(52) U.S. Cl. CPC A61K 36/185; A61K 8/9789; A61Q 19/00; A61K 2236/15; A61K 2236/333; A61K 2236/53; A61K 2800/10; A61K 2800/30

(57) ABSTRACT

The current invention relates to a method for extracting biological material for obtaining cosmetic and/or pharmaceutical components in the obtained extract using an extraction solvent, comprising the steps of contacting the biological material with the extraction solvent, immersing the biological material in the extraction solvent, macerating or percolating or infusing the mixture, filtering and/or centrifuging the extraction product obtained, thereby obtaining a liquid extract, wherein said extraction solvent comprises water and one or a plurality of alkane diols with 5 through 12 carbon atoms. The invention also relates to a cosmetic preparation and use of a composition.

### FIELD OF THE INVENTION

The present invention relates to a method for extracting biological material. In a second aspect, the present invention relates to a cosmetic preparation. In another aspect, the present invention also relates to a use for extracting compounds and of the extracted compounds.

### BACKGROUND

Cosmetic formulation involves a wide range of ingredients (actives and auxiliaries, fragrance, pigments, oils, emollients, surfactants, preservatives, texturing agents, etc.). Bioactive ingredients take thus a very special part in that landscape in order to ensure the final activity of cosmetic products (antioxidant, anti-aging, photo-protection, moisturization, etc.). Over the last decades, the aversion towards synthetic additives and products generated a great interest and a growing demand for natural extracts. This general trend has prompted attempts to exploit natural extracts and ingredients given their richness in bioactive substances. Biological materials such as plants have long been a source of inspiration for the cosmetics industry. Particularly, plant materials have been increasingly investigated as sources of natural active ingredients including primary and secondary metabolites such as polyphenols, essential oils, or even pigments. However, plants metabolites are complex mixtures generally present at low concentrations. Before such substances can be used, they have to be extracted from their containing matrices.

Various methods can be used for that purpose. Conventionally, such extracts are obtained using solid/liquid extraction. In KR101207557 the extraction solvent is selected from the group consisting in water, alcohol of 1 to 4 carbon atoms, acetone, ethyl acetate, butyl acetate or 1,3-butylene glycol, and their mixtures. The extraction solvent is subsequently removed by heating for 3 to 20 hours at 40˜100° C., or for 1 to 15 days at 4˜40° C. by a rotary vacuum evaporator.

This known method is often time and energy consuming, induces the use of huge amounts of water or petroleum solvents harmful to the environment and users, and generates large quantities of waste. Resulting extracts are not always safe as they may contain residual solvents, contaminants from raw materials, or denatured compounds due to drastic extraction conditions.

Vieira, Vanessa, et al. (“Insights on the extraction performance of alkanediols and glycerol: Using Juglans regia L. leaves as a source of bioactive compounds.” Molecules 25.11 (2020): 2497) describe solvents to extract phytochemicals from plant material. Vieira, Vanessa, et al. investigate water, ethanol, glycerol, 1,2-ethanediol, 1,2-propanediol, 1,3-propanediol, 1,3-butanediol, 1,2-pentanediol, 1,5-pentanediol and 1,2-hexanediol.

KR 2016 0054055 relates to a food composition for preventing and improving diabetes. The extract is extracted with at least one extraction solvent selected from water, a lower alcohol having 1 to 4 carbon atoms, a polyhydric alcohol, or a mixture thereof.

JP 2000 044419 describes a method for producing a plant extract. The extract is added to a skin care preparation.

The present invention aims to resolve at least some of the problems and disadvantages mentioned above. The aim of the invention is to provide a method which eliminates those disadvantages.

### SUMMARY OF THE INVENTION

In a first aspect, the present invention and embodiments thereof serve to provide a solution to one or more of above-mentioned disadvantages. To this end, the present invention relates to a method for extracting biological material, according to claim 1. The extraction solvent is a clear, stable and fluid mixture comprising water and a C5-C12-diol. Preferred embodiments of the method are shown in any of the claims 2 to 10. Preferably the extraction solvent is pentylene diol and more preferably a pentylene glycol of natural origin. According to the method, exceptionally stable extracts are obtained with a high content of extracted components due to the superior dissolving and extracting properties of the chosen solvent.

In a second aspect, the present invention relates to a cosmetic and/or pharmaceutical preparation, according to claim 12. Preferred embodiments of the method are shown in the claims 13 and 14. Conventionally used solvents are petroleum-based solvents and comprise Volatile Organic Compounds (VOC) which are harmful for human health and the environment. According to this invention these solvents are replaced by safer and more sustainable alternatives.

In a third aspect the present invention relates to a use according to claim 15. The use as described herein provides the advantageous effect that the obtained extracts are characterized by being readily suitable as ingredients for cosmetics, personal care or pharmaceutical formulations. No evaporation step, to up-concentrate the extracted compounds, is deemed to be required.

### DESCRIPTION OF FIGURES

The following description of the figures of specific embodiments of the invention is merely exemplary in nature and is not intended to limit the present teachings, their application or uses.

FIG. 1 shows experimental solubilities of Hesperidin and Gallic Acid compared to values predicted by COSMO-RS, according to an embodiment of the invention.

FIG. 2 shows a ternary phase diagram of PG/water/CG according to an embodiment of the invention, wherein the different points 1-6 in the diagram refer to the mixtures used for the solubility tests.

FIG. 3 shows an example of a ternary phase diagram, according to an embodiment of the invention.

FIG. 4 shows ternary phase diagrams with oxygenated monoterpenes: PG/Water/Carvacrol (A), PG/Water/Thymol (B), PG/Water/Carvone (C) and PG/Water/Geraniol (D), according to an embodiment of the invention.

FIG. 5 shows ternary phase diagrams with non-oxygenated monoterpenes: PG/Water/Limonene (A), PG/Water/α-Pinene (B) and PG/Water/α-Terpinene (C), according to an embodiment of the invention.

FIG. 6 shows a ternary phase diagram: PG/Water/Non-oxygenated monoterpenes (A), PG/Water/Oxygenated monoterpenes (B) based on calculated average values, according to an embodiment of the invention.

FIG. 7 shows a ternary phase diagram: PG/Water/Thyme oil (A), PG/Water/Citronella oil (B), PG/Water/Eucalyptus oil (C) and PG/Water/Lavender oil (D), according to an embodiment of the invention.

FIG. 8 shows a ternary phase diagram: PG/Water/Plum kernel oil (A), PG/Water/Hemp seed oil (B), PG/Water/Pomegranate seed oil (C), according to an embodiment of the invention.

FIG. 9 shows ternary phase diagrams at ambient temperature in weight fractions of the systems water/PG/EtOH, water/PG/2-MeOx, water/PG/CG and water/PG/hexane, according to an embodiment of the invention.

### DETAILED DESCRIPTION OF THE INVENTION

Solvents occupy a strategic place in the development of the concept of green chemistry and green extraction. Consequently, experts in the field are expected to find new “green” alternative solvents in replacement of petrochemical solvents. As of today, efficient extraction solvents are often still of petrochemical origin. These conventional solvents present some advantages related especially to their relatively low boiling point and low enthalpy of vaporization, which facilitates their removal after extraction and limits the energy consumption related to this step. Moreover, some of them are very selective towards targeted molecules, such as hexane for the extraction of lipophilic molecules.

Nonetheless, besides their obvious technical value, petrochemical solvents also present negative features. Petroleum-sourced solvents are obtained from non-renewable resources, are often Volatile Organic Compounds (VOC) and harmful to human health and environment. They are thus more and more replaced by more renewable, safer, and more sustainable alternatives.

In this context, a growing aversion towards petrochemical solvents emerged, particularly in the cosmetic industry. The increase of strengthened legislation regarding cosmetic ingredients and solvents further forced industrials and academics to find potential replacement solvents.

A problem often encountered by the person skilled in the art is that of the extremely high polarity of water, thus limiting its use to hydrophilic molecules. Therefore, non-polar and some semi-polar compounds cannot be extracted using water.

In this context, there is a demand for alternative solvents able to solubilize and then to extract molecules with a larger spectrum of polarity. To the best of our knowledge, there are no reports on appropriate solvents that meet the criteria of green extraction and naturalness without sacrificing dissolving and extractant abilities. Hence, the relevance of the present invention lies in the potential of bio-based 1,2-alkanediols, initially known as cosmetic ingredients, for dissolving and extracting natural compounds destined to the cosmetic field.

The present invention relates to:
The formulation of a new green solvent comprising at least one preferably bio-based 1,2-alkanediol with carbon chain of between 5 and 12 carbon atoms in a mixture with water, said 1,2-alkanediol being preferably bio-based.
Bio-based Pentylene Glycol (PG) mixed with water in a ratio from 0.01 to 99.99 wt %.
The use of ‘Green’ Pentylene Glycol (PG) as (i) natural functional cosmetic ingredient based on its moisturizing and stabilizing properties and at the same time as (ii) as bio-based green solvent with interesting dissolving and extracting abilities.
The use of Pentylene Glycol (PG) and/or bio-based Caprylyl Glycol (CG) as extracting solvent.
A method for solid/liquid extraction using the said novel green solvent, optionally combined with size reduction and process intensification techniques such as ultrasound, microwave, as well as combinations thereof.
The liquid extract derived from the implementation of the said method using the said aqueous Pentylene Glycol (PG) to develop a self-preserved multi-functional system which has interesting biological activities.
The said multi-functional system is based on the combination of the defined solvent with extracted natural active compounds.
A preferred embodiment of the invention is the use of aqueous PG which is useful in providing improved dissolving and extraction properties of natural compounds contained in biological material.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

As used herein, the skilled person would understand the term “plant” to mean a living organism of the kind exemplified by trees, shrubs, herbs, grasses, ferns, and mosses, typically growing in a permanent site, absorbing water and inorganic substances through its roots, and synthesizing nutrients in its leaves by photosynthesis using pigment chlorophyll. For example, the term plants may refer to flowers and vegetables.

As used herein, solid/liquid extraction is the selective release, washing, or leaching of substances from solids using liquid solvents. Complex multicomponent mixtures are obtained from solid-liquid extraction processes. One or more subsequent purification steps are required to achieve an enriched botanical extract or a pure substance.

As used herein, the skilled person would understand that the term “grind” may mean that the solid material had been subjected to mechanical forces to crush, pulverize, or reduce to powder by friction prior to being immersed in the solvent. The material can be biological or not. Two levels of grinding were performed in the present invention: (i) coarse grinding for particles size bigger than 400 μm and (ii) fine grinding for particles size smaller than 100 μm. As used herein, the skilled person would understand that the term “unground” may mean that the biological material had not been subjected to any mechanical forces, other than manual cutting, prior to being immersed in the solvent.

“Bio-based” means that the organic compound is synthesized from biologically produced organic components. “Biologically based”, and “green”; “biologically derived”; “produced from renewable resources”; and “bio-derived” can be used synonymously herein. Virtually all forms of life on Earth depend on production of organic molecules by plants or other autotrophs to produce the chemical energy that facilitates growth and reproduction. Therefore, the carbon that exists in the atmosphere becomes part of all life forms, and their biological products. These renewably based organic molecules that biodegrade to CO2 do not contribute to global warming as there is no net increase of carbon emitted to the atmosphere. In contrast, fossil fuel-based carbon does not have the signature radiocarbon ratio of atmospheric carbon dioxide. Assessment of the renewably based carbon in a material can be performed through standard test methods. Using radiocarbon and isotope ratio mass spectrometry analysis, the biobased content of materials can be determined. ASTM International, formally known as the American Society for Testing and Materials, has established a standard method for assessing the biobased content of materials. The ASTM method is designated ASTM-D6866. The application of ASTM-D6866 to derive a “biobased content” is built on the same concepts as radiocarbon dating, but without use of the age equations. The analysis is performed by deriving a ratio of the amount of radiocarbon in an unknown sample to that of a modem reference standard. The ratio is reported as a percentage with the units “pMC” (percent modern carbon). If the material being analyzed is a mixture of current radiocarbon and fossil carbon, then the pMC value obtained correlates directly to the amount of biomass material present in the sample. In this regard, a sample measuring at least 99 pMC will be considered to be fully bio-based.

“Biological material” means organic compounds produced by one or more species or strains of living organisms, including particularly plants, animals, fungi, algae, protozoa, bacteria, yeasts, and other prokaryotes.

“C5-C12 alkanediols” or “alkane diols with 5 through 12 carbon atoms” include 1,2 alkane glycols such as pentylene (C5) glycol, 1,2-hexanediol (C6), 1,2-heptanediol (C7), caprylyl (C8) glycol, 1,2-nonanediol (C9), decylene (C10) glycol, undecylene (C11) glycol, lauryl (C12) glycol, but also 1,3 C5-C12 alkanediols, 1,4 C5-C12 alkane diols, 1,5 C5-C12 alkane diols, 1,6 C6-C12 alkane diols, 2,3 C5-C12 alkane diols, 2,4 C6-C12 alkane diols, etc.

“C3-C18 glycols” are propylene (C3) glycol, butylene (C4) glycol, pentylene (C5) glycol, 1,2-hexanediol (C6), 1,2-heptanediol (C7), 1,2-octanediol (C8), 1,2-nonanediol (C9), decylene (C10) glycol, undecylene (C11) glycol, lauryl (C12) glycol, tridecylene (C13) glycol, tetradecylene (C14) glycol, pentadecylene (C15) glycol, cetyl (C16) glycol, heptadecylene (C17) glycol and stearyl (C18) glycol. Said glycols include isomers and branched or unbranched alternatives thereof.

In a first aspect, the invention relates to a method for extracting biological material for obtaining cosmetic and/or pharmaceutical components in the obtained extract using an extraction solvent, comprising the steps of contacting the biological material with the extraction solvent, immersing the biological material in the extraction solvent, macerating or percolating or infusing the mixture, filtering and/or centrifuging the extraction product obtained, thereby obtaining a liquid extract, wherein said extraction solvent comprises water and one or a plurality of alkane diols with 5 through 12 carbon atoms (C5-C12). In a further embodiment, the alkane diol is a 1,2 diol, preferably with 5 through 10 carbon atoms. In a further embodiment, the alkane diol is pentylene glycol (1,2-pentanediol). In a further embodiment, the extraction solvent and the biological material are used with a weight ratio of between 1:1 and 100:1, preferably between 2:1 and 100:1, more preferably between 5:1 and 20:1, most preferably at a ratio of 10:1.

In a further embodiment, the biological material and the solvent are heated, preferably to 30-100° C., more preferably to 50-70° C. and most preferably to 55-65° C. In a further embodiment, the biological material is submitted to shredding and/or grinding, prior to extraction, to an average particle size (D50) below 1 cm and more preferably below 1 mm. In a further embodiment, the extraction is ultrasound-assisted preferably with a frequency between 10-40 kHz and more preferably 20-25 kHz and/or microwave-assisted with a frequency between 300 MHz and 300 GHz. In a further embodiment, the weight percentage of the alkane diol with 5 through 12 carbon atoms in water ranges from 0.01 to 99.99%, preferably from 10 to 90%. In a further embodiment, the extraction solvent further comprises one or more additional alkane diols with 3 through 18 carbon atoms (C3-C18-diol), preferably C3-C18 1,2-diol, more preferably C4-C12 1,2-diol and most preferably 1,2-octanediol (caprylyl glycol). In a further embodiment, the C5-C12 and/or the C3-C18-diol are produced from renewable resources. In a further embodiment, the biological material can be a plant or part or parts thereof; an animal and/or prokaryote or part or parts thereof. In a further embodiment, the plant biological material is selected from the group comprising cherry blossom, horsetail, plantain, saffron flowers, verbena leaves, rose of Jerico, rosemary, algae, fungi, Selaginella pulvinata, Tillandsia usnoides, citrus, Matricaria, iris, olive or other Embryophyta; the animal material is preferably selected from invertebrates; and prokaryotic biological material is preferably selected from gram-negative or gram-positive bacteria.

The invention relates to a method for extracting biological material for obtaining cosmetic and/or pharmaceutical components in the obtained extract using an extraction solvent, comprising the steps of contacting the biological material with the extraction solvent, immersing the biological material in the extraction solvent, macerating or percolating or infusing the mixture, filtering and/or centrifuging the extraction product obtained, thereby obtaining a liquid extract, wherein said extraction solvent comprises water and one or a plurality of C5-C12 alkanediols.

The inventors unexpectedly observed that C5-C12 alkane diols are efficient in extracting desired compounds from biological material. Said C5-C12 alkane diols were so efficient that the use of conventional solvents such as hexane or ethanol could be significantly reduced or even completely replaced by such C5-C12 alkane diols. Said C5-C12 alkane diols yielded an extraction product more suitable as an aqueous cosmetic solution. Less downstream processing and purification is required as such extraction solvents can be used in cosmetic end products.

In an embodiment, the C5-C12 alkanediols are 1,2 C5-C12 alkane diols. In an embodiment, the C5-C12 alkanediols are 1,3 C5-C12 alkane diols. In an embodiment, the C5-C12 alkanediols are 2,3 C5-C12 alkane diols. In an embodiment, the C5-C12 alkanediols are 1,4 C5-C12 alkane diols. In an embodiment, the C5-C12 alkanediols are 1,5 C5-C12 alkane diols.

In an embodiment, said extraction solvent comprises 10-75 wt. % pentylene glycol, preferably said extraction solvent comprises 25-70 wt. % pentylene glycol. In an embodiment, said extraction solvent comprises 40-75 wt. % pentylene glycol, preferably said extraction solvent comprises 50-75 wt. % pentylene glycol. In an embodiment, said extraction solvent comprises 40-95 wt. % pentylene glycol, preferably said extraction solvent comprises 50-90 wt. % pentylene glycol.

Experiments showed a better extraction of desired compounds such as Gallic acid, Rosmarinic Acid, Hesperidin, Curcumin, or Ferulic Acid when the extraction solvent is not 100% water but contains some pentylene glycol. However, extraction with pure pentylene glycol and no water did also not show the best results. Surprisingly a mixture of both water and pentylene glycol demonstrated synergistic results and higher solubility of the desired compounds. “Pentylene glycol”, “PG” and “1,2-pentanediol” refer to the same compound.

In an embodiment, said extraction solvent essentially consists of:
one or a plurality of C5-C12 alkanediols, wherein the total amount of C5-C12 alkanediols is 10-70 wt. % of the extraction solvent, preferably 25-70 wt. % of the extraction solvent; and
water, for the remainder of the extraction solvent.

Said C5-C12 alkane diols were shown to be very efficient and conventional solvents such as hexane or ethanol could be completely replaced by a mixture of C5-C12 alkane diols. Said C5-C12 alkane diols yielded an extraction product more suitable as an aqueous cosmetic solution. Less downstream processing and purification is required as such extraction solvents can be used in cosmetic end products. All traces of conventional solvents should be removed from the extract before they can be used in cosmetic products. Many C5-C12 alkane diols have demonstrated positive effects for the skin and are desirable in many cosmetic products. It follows that, said C5-C12 alkane diols should not be removed from the extraction product before further processing.

In an embodiment, said extraction solvent essentially consists of:
pentylene glycol, in an amount of 10-70 wt. % of the extraction solvent, preferably in an amount of 25-70 wt. % of the extraction solvent; and
water, for the remainder of the extraction solvent.

In an embodiment, said extraction solvent comprises, preferably said extraction solvent essentially consists, of:
a first C5-C12 alkanediol, preferably said first C5-C12 alkanediol is pentylene glycol;
a second C5-C12 alkanediol, preferably said second C5-C12 alkanediol is caprylyl glycol; and
water.

In an embodiment, said extraction solvent comprises, preferably said extraction solvent essentially consists, of: pentylene glycol, caprylyl glycol and water. In an embodiment, said extraction solvent comprises, preferably said extraction solvent essentially consists, of: 10-70 wt. % pentylene glycol; 1-50 wt. % caprylyl glycol; and water, for the remainder of the extraction solvent. “Caprylyl Glycol”, “CG” and “1,2-octanediol” refer to the same compound.

A mixture of pentylene glycol, caprylyl glycol and water showed an improved solubility of Hesperidin and Curcumin compared to the mixtures consisting of pentylene glycol and water. In some cases, a second C5-C12 alkanediol further improves the solubility of a desired compound. The additional synergistic effect was unexpected.

In an embodiment, the biological material is selected from: Rosmarinus, Verbena, Cistus ladanifer, Matricaria chamomilla and Iris germanica. In an embodiment, the biological material is selected from a species of the family of the: Lamiaceae, Verbenaceae, Cistaceae, Asteraceae and Iridaceae.

Rosmarinus, Verbena, Cistus ladanifer, Matricaria chamomilla and Iris germanica comprise several desired compounds for cosmetic extracts. Said compounds are suitable to be extracted in an efficient manner using a C5-C12 alkanediol.

In an embodiment, the extraction solvent essentially consists of 20-40 wt. % 1,2-pentanediol and water and the biological material is Cistus ladanifer, wherein the weight ratio of the biological material contacted with the extraction solvent is 1:1-1:100, preferably 1:10. In an embodiment, the extraction solvent essentially consists of 20-40 wt. % 1,2-pentanediol, 5-10 wt. % 1,2-octanediol and water and the biological material is Cistus ladanifer, wherein the weight ratio of the biological material contacted with the extraction solvent is 1:1-1:100, preferably 1:10. In an embodiment, the method according to this invention utilizes an extraction solvent essentially consisting of 20-40 wt. % 1,2-pentanediol and water; and a mixture of Cistus ladanifer and said extraction solvent was macerated at 60-80° C. for 15-100 min and subsequently filtered. Said method is highly suitable for the extraction of Apigenin, ellagitannins and kaempherol glucoside. Extraction of Apigenin, ellagitannins and kaempherol glucoside with one or more C5-C12 alkanediols is preferred over extraction with ethanol, or hexane mixtures.

The inventors have unexpectedly observed that a mixture comprising water and a C5-C12-diol show very good extraction properties. Medium-chain 1,2-alkanediols such as bio-based pentylene glycol (PG), combined with water, can act as efficient solvents for natural solubilization and extraction. By virtue of their amphiphilic nature, these eco-sourced alkanediols represent an excellent alternative to water or lower-chain alcohols. Indeed, they are expected to enlarge the spectrum of polarity while respecting requirements for bio-based and sustainable origin. Furthermore, natural extracts can be stabilized by this novel combination of natural cosmetic solvents. Ratios of the two components (water and one or more C5-C12-diols) can be altered to improve extraction characteristics. Via heating or grinding, using ultrasounds or microwaves the concentrations of the extracted compounds can be further adjusted as desired.

In an embodiment, said extraction solvent essentially consists of water and 10-80 wt. % of one or a plurality of C5-C12 alkane diols. In an embodiment, said extraction solvent essentially consists of water and 10-70 wt. % of one or a plurality of C5-C12 alkane diols, preferably 20-70 wt. %, more preferably 30-70 wt. %, more preferably 30-60 wt. %, most preferably 30-50 wt. %. In an embodiment, said extraction solvent essentially consists of 10-70 wt. % of a plurality of C5-C12 alkane diol and water. In an embodiment, said extraction solvent essentially consists of 10-70 wt. % of one or a plurality of C5-C12 alkane diols and water. In an embodiment, said extraction solvent essentially consists of 20-60 wt. % of one or a plurality of C5-C12 alkane diols and water. In an embodiment, said extraction solvent essentially consists of water and 10-70 wt. % of one or a plurality of C5-C12 alkane diols, preferably 10-60 wt. %, more preferably 10-50 wt. %, and most preferably 15-45 wt. %.

In an embodiment, said extraction solvent essentially consists of a C5-C12 alkane diol, water and a C3-C18 alkane diol, preferably a C5-C10 alkane diol, water and a C5-C18 1,2-diol, more preferably a C5-C10 alkane diol, water and a C5-C12 1,2-diol and most preferably 1,2-pentylene glycol, water and 1,2-octanediol.

In an embodiment, the extraction solvent further comprises an additional C3-C18-diol, preferably a C5-C18 diol, more preferably a C5-C14 diol, more preferably a C5-C14 alkane 1,2-diol, and more preferably 1,2-octanediol.

In an embodiment, said extraction solvent essentially consists of a C5-C12 alkane diol, water and a C3-C18-diol, wherein the weight ratio (weight/weight) of the C5-C12 alkane diol and the C3-C18-diol is between 1:1 and 100:1, preferably between 1:1 and 1:50, more preferably 1:1 and 10:1. In a further embodiment, the C5-C12 alkane diol and the C3-C18-diol are, respectively, a C5-C10 alkane diol and a C3-C12 alkane diol, more preferably a C5-C8 alkane 1,2-diol and a C5-C12 alkane 1,2-diol, even more preferably 1,2-pentylene glycol and 1,2-octanediol.

In an embodiment, said extraction solvent essentially consists of a C5-C12 alkane diol, water and a C3-C18-diol, wherein the weight ratio (weight/weight) of the C3-C18-diol and the C5-C12 alkane diol is between 1:1 and 100:1, preferably between 1:1 and 1:50, more preferably 1:1 and 10:1. In a further embodiment, the C5-C12 alkane diol and the C3-C18-diol are, respectively, a C5-C10 alkane diol and a C3-C12 alkane diol, more preferably a C5-C8 alkane 1,2-diol and a C5-C12 alkane 1,2-diol, even more preferably 1,2-pentylene glycol and 1,2-octanediol.

In an embodiment, said extraction solvent essentially consists of a C5-C12 alkane diol, water and a second alkane diol with a longer chain length, preferably said extraction solvent essentially consists of a C5-C10 alkane diol, water and the second alkane diol with a longer chain length, more preferably of a C5-C10 alkane 1,2-diol, water and a second alkane 1,2-diol with a longer chain length, most preferably of 1,2-pentylene glycol, water and a second alkane 1,2-diol with a longer chain length

In an embodiment, said extraction solvent essentially consists of 10-70 wt % of a C5 alkane diol, 10-70 wt % of water and 10-70 wt % of a C6 alkane diol. In an embodiment, said extraction solvent essentially consists of 10-70 wt % of a C5 alkane diol, 10-70 wt % of water and 10-70 wt % of a C7 alkane diol. In an embodiment, said extraction solvent essentially consists of 10-70 wt % of a C5 alkane diol, 10-70 wt % of water and 10-70 wt % of a C8 alkane diol. In an embodiment, said extraction solvent essentially consists of 10-70 wt % of a C5 alkane diol, 10-70 wt % of water and 10-70 wt % of a C9 alkane diol. In an embodiment, said extraction solvent essentially consists of 10-70 wt % of a C5 alkane diol, 10-70 wt % of water and 10-70 wt % of a C10 alkane diol. In an embodiment, said extraction solvent essentially consists of 10-70 wt % of a C5 alkane diol, 10-70 wt % of water and 10-70 wt % of a C11 alkane diol. In an embodiment, said extraction solvent essentially consists of 10-70 wt % of a C5 alkane diol, 10-70 wt % of water and 10-70 wt % of a C12 alkane diol. In an embodiment, said extraction solvent essentially consists of 10-70 wt % of a C5 alkane diol, 10-70 wt % of water and 10-70 wt % of a C13 alkane diol. In an embodiment, said extraction solvent essentially consists of 10-70 wt % of a C5 alkane diol, 10-70 wt % of water and 10-70 wt % of a C14 alkane diol. In an embodiment, said extraction solvent essentially consists of 10-70 wt % of a C5 alkane diol, 10-70 wt % of water and 10-70 wt % of a C15 alkane diol. In an embodiment, said extraction solvent essentially consists of 10-70 wt % of a C5 alkane diol, 10-70 wt % of water and 10-70 wt % of a C16 alkane diol. In an embodiment, said extraction solvent essentially consists of 10-70 wt % of a C5 alkane diol, 10-70 wt % of water and 10-70 wt % of a C17 alkane diol. In an embodiment, said extraction solvent essentially consists of 10-70 wt % of a C5 alkane diol, 10-70 wt % of water and 10-70 wt % of a C18 alkane diol. In a further embodiment, each of the compounds is present in a concentration of 20-60 wt. %, preferably 20-50 wt. %, more preferably 20-40 wt. %.

In an embodiment, said extraction solvent essentially consists of 10-70 wt % of a C8 alkane diol, 10-70 wt % of water and 10-70 wt % of a C10 alkane diol. In an embodiment, said extraction solvent essentially consists of 10-70 wt % of a C8 alkane diol, 10-70 wt % of water and 10-70 wt % of a C11 alkane diol. In an embodiment, said extraction solvent essentially consists of 10-70 wt % of a C8 alkane diol, 10-70 wt % of water and 10-70 wt % of a C12 alkane diol. In an embodiment, said extraction solvent essentially consists of 10-70 wt % of a C8 alkane diol, 10-70 wt % of water and 10-70 wt % of a C13 alkane diol. In an embodiment, said extraction solvent essentially consists of 10-70 wt % of a C8 alkane diol, 10-70 wt % of water and 10-70 wt % of a C14 alkane diol. In an embodiment, said extraction solvent essentially consists of 10-70 wt % of a C8 alkane diol, 10-70 wt % of water and 10-70 wt % of a C15 alkane diol. In an embodiment, said extraction solvent essentially consists of 10-70 wt % of a C8 alkane diol, 10-70 wt % of water and 10-70 wt % of a C16 alkane diol. In an embodiment, said extraction solvent essentially consists of 10-70 wt % of a C8 alkane diol, 10-70 wt % of water and 10-70 wt % of a C17 alkane diol. In an embodiment, said extraction solvent essentially consists of 10-70 wt % of a C8 alkane diol, 10-70 wt % of water and 10-70 wt % of a C18 alkane diol. In a further embodiment, each of the compounds is present in a concentration of 20-60 wt. %, preferably 20-50 wt. %, more preferably 20-40 wt. %. A combination of two alkane diols and water showed a better solubility of several desirable compounds compared to other extraction solvents.

In an embodiment, said C5-C12 alkanediols are produced from renewable resources, preferably said C5- to C12 alkanediols are biobased. Biobased alternatives are desired over petrochemical alternatives. The impurities are less toxic or damaging for the skin. The presence of certain organic compounds in cosmetic products can significantly reduce the effectiveness of said cosmetic product.

For the production of bio-based PG, squeezed sugar cane, i.e. bagasse, a waste product of sugar production, can be used. Such plant residues contain sugar monomers with five carbon atoms, linked together in the form of the indigestible polymer hemicellulose. The same type of sugar polymers can also be found in other lignocellulosic biomasses such as, for example, date-palm trees, sorghum straw, corncobs, wood, oat hulls, rice hull and rice straw. By splitting the polymeric hemicellulose with water into its components (hydrolysis) and by eliminating water from the resulting monomers xylose and arabinose (dehydration), the raw material “furfural” can be obtained in a single technical step. This intermediate compound is isolated by steam distillation and can be further used as a starting material for various applications. Bio-based PG is obtained by reacting the raw material furfural with hydrogen via catalytic hydrogenation.

It should be mentioned that solvent polarity and chemical affinity are not the only requirements for efficient plant extraction. The inherent characteristics of biomaterials need to be taken into consideration. Prior treatment such as size reduction may have a considerable impact on extraction performance. Such a grinding step is intended to enhance the solid/liquid contact and solvent penetration into structures containing active targeted compounds.

It has been unexpectedly found that aqueous 1,2-alkanediols enhanced significantly the solubility of molecules of interest with a large spectrum of polarity such as the one encompassed by the model substances namely hesperidin, gallic acid, rosmarinic acid, ferulic acid and curcumin. Aqueous solutions, at defined proportions of 1,2-alkanediol, make it possible to solubilize high quantities of the studied molecules compared to reference extraction solvents (water and hydro-alcoholic mixture).

Hence, results interestingly proved that the developed bio-based solvent was suitable as a natural solubilizing agent. This invention is all the more significant in that until now industry is still trying to find bio-based alternatives as efficient as petrochemical-derived solvents.

Among all tested solvents, mixtures of water and aqueous 1,2-alkanediol meet best the greenness and naturalness requirements in terms of their sourcing, availability, biodegradability, environmental footprint, etc. This invention discloses thus a bio-based solvent with excellent dissolving properties.

In an embodiment, the present invention provides a method for extracting natural biological compounds including the following steps:
a. formulating aqueous solutions of preferably bio-based 1,2-alkanediol at a defined proportion of 1,2-alkanediol
b. immersing, while stirring, a ground or unground biological material in the extraction solvent according to the invention, then
c. macerating or optionally submitting solid/liquid mixture obtained in step b. to ultrasound, microwave or combined ultrasound/microwave at a temperature between 1 and 105° C., preferably between 40 and 80° C., more preferably between 55 and 65° C., then
d. separating the liquid extract from solid compartments, thereby obtaining a liquid extract derived from the plant (e.g. vegetable) and/or animal and/or prokaryotic biological material.

The extraction product obtained in b. may be filtered in step c. using any techniques known to those skilled in the art, such as but not limited to filtration or centrifugation.

Thus, in a further embodiment, the invention also provides a liquid extract, preferably a bio-based extract, comprising plant (e.g. vegetable) and/or animal and/or prokaryotic biological material obtained or obtainable by the extraction method or use previously defined.

The extracted compounds comprise phenolic acids and esters, flavonoids, phenolic alcohols, antioxidants, carotenoids, alkaloids, lipids, phenylpropanoids, flavors, fragrances, biocides, antimicrobials, proteins, enzymes, colorants, pigments, surfactants and terpenoids including saponins, from plant (e.g. vegetable) and/or animal and/or prokaryotic biological material, and preferably from plant (e.g. vegetable) biological material.

The extractable compounds according to the present invention non-exhaustively include terpenes, terpenoids, flavones and flavonoids, steroids, sterols, saponins and sapogenins, alkanes, alkaloids, amines, amino acids, aldehydes, irrridoids, phenylpropanoids, alcohols, polyols, lipids, fatty acids, lignans, phenols, pyrones, butenolides, lactones, chalcones, ketones, benzenes, cyclohexanes, glucosides, glycosides, cyanidines, furans, phorbols, quinones and phloroglucinols in aglycone form or, where appropriate, in glycosylated form. The invention can also be applied to the extraction of high molecular weight bioactive molecules such as proteins, peptides, enzymes, polysaccharides, oligosaccharides and carbohydrates. By way of example, the extractable compounds according to the present invention include in a non-exhaustive manner the following substances: catechin, epicatechin, catechin gallate, epicatechin gallate, gallocatechin, epigallocatechin, gallocatechin gallate, epigallocatechin gallate, catechic tannins, gallic tannins, rhamnetin, fisetin, robinetin, gossypetin, orientin, homoorientine, cirsiliol, homoprotocatechuic acid, dihydrocaffeic acid, ethyl ester of protocatechuic acid, propyl gallate, gallic acid, protocatechuic acid, caffeic acid, rosmarinic acid, esculetylin, 4-methicculetin, neochlorogenic acid, phenethyl ester of caffeic acid, chicoric acid, echinacoside, beta-D-glucopyranoside, verbascoside, hydroxytyrosol, maclurine, 3,4-dihydroxybenzaldehyde, 3,4-dihydroxybenzophenone, butein, 3,4-dihydroxyacetophenone, seain, eriodictyolchalcone, pyrocatechol, nordihydroguaiaretic acid, 3-hydroxydaidzein, oleuropein, marit imein, salicylic acid, myristicin, eugenol, umbelliferone, aesculetin, juglone, resveratrol, kaempferol, afzeline, astragalin, juglanin, robinin, trifoline, kaempferol-3-O-rutinoside, daidzine, puerarin, diarimmyanolines, procinephyanidins marmarin, marmesin, angelicin, imperatorin, xanthotoxin, bergapten, psoralen, linalool, 1,8-cineole, alpha-pinene, beta-sitosterol, campestrol, stigmasterol, fruit acids (lactic acid, glycolic acid, malic acid, citric acid, etc.), vitamin E (tocopherols), vitamin A (retinoids), provitamin A (carotenoids), vitamin C, B vitamins (thiamine, biotin, nicotinamide, panthothenic acid, etc.), camphor, menthol, myrcene, abyssinone I, abyssinone V, afzelechin, ampelopsin, aromadendrine, auriculoside, broussine, broussonin C, butine, butrin, davidigenine, diffutin, 7,4′-dihydroxylflavane, 2,6-dihydroxyl-4′-methoxydihydro-chalcone, 7,3′-dihydroxyl-4-methoxy-8-methylflavane, 7,4′-dihydroxyl-8-methylfalvane, 6,8-diprenylnaringenin, dracorubin, eriocitrin, eriodictyol, farrerol, fisetinidol, fisetinidol-4-ol, fustine, garbanzol, glabranin, glepidotin β, glycyphylline, hesperetine, hesperidin, homoeriodictaniclavine, hesperanetolaejlavine, 7-is-isperanetolaejlavinlavocafictan, isperanetolaejlavinlavocafourone, 7-isperanetolaejlavocafictan, liquiretigenine, manniflavanone, methoxytaxifoline, narirutin, neoastilbine, neoeriocitrin, neohesperidine, phloretin, phellamurine, phloridzine, pinobanksin, pinocembrin, pinocembrin 7-rhamnosily-glucoside, sakopanchurin, sakopinosyl-glucosine, sakopanchurin, sristopanchurin, sristopanchlorin, sristopidylchlorin, sristopidin, sristopidylchlorin, sristopidylchlorin, sicopinyl-sidrin, sristopanchurin, sicopinyl-glucan strobopinin, taxifolene, taxifolin-3-O-acetate, tephrowatsine, theasinensin A, uvaretine, naringenin, naringenin glycosides, oglucosylrutin, alpha-glucosylmyricetine, alpha-glucosylisoquercetin, alpha-glucosylquercetin, 5 ‘(hesperidin) (3-glucosylquercetine, hesperidin, 7-trihydroxy-4’-methoxyflavanone 7-rhamnoglucoside, hesperitine 7-O-rhamnoglucoside), neohesperidine, rutin (3,3′,4′,5,7-pentahydroxyflavone 3-rhamnoglucoside, quercetin 3-rhamnoglucoside), diosmin (3′,4′,7-trihydroxy-5-methoxyflavanone 7-rhamnoglucoside), eriodictine, apigenin 7-glucoside (4′,5,7-trihydroxyflavone 7-glucoside), kaempferol, quercitrin, avicularin, myricitin.

In an embodiment, the plant is immersed or heated to reflux together with an extraction solvent, and then the resultant may be appropriately subjected to filtration, concentration, freeze-drying or the like, to thereby obtain a concentrated extract, a dried powder or the like. In an embodiment, examples of the extraction solvent may include the generally-used organic solvents such as methanol, ethanol, propanol, butanol, ether, ethylene glycol, propylene glycol, butylene glycol, petroleum ether, hexane, heptane, cyclohexane, ethyl acetate, acetone, toluene, dichloroethane and chloroform, water, and the like, and these may be used as mixtures of one or more species. These mixtures may further comprise alkane diols with 5 through 12 carbon atoms (C5-C12), preferably pentylene glycol. The extraction treatment can be carried out according to a conventional method, usually at a temperature of about 3 to 100º C for several hours to several weeks, and the extract can be used after being purified by gel filtration, column chromatography, fractional distillation or the like. In an embodiment, enzymes or other catalysts can be added to improve extraction efficiency.

Solid processing prior to extraction must be taken into account to design an efficient extraction process. According to this invention, coarse grinding, with an average particle size (D50) above 400 μm, appeared as the most suitable prior treatment. Solid/liquid extraction from coarsely ground plant materials provided the best extraction performances particularly in terms of total phenol content and total content of extracted components.

In an embodiment, the extraction process consists of maceration of a ground or unground biological material immersed in aqueous green PG at a temperature between 50 and 80° C. and preferably 60° C. with stirring between 10 minutes and 3 hours, preferably between 30 and 120 minutes and preferably for about 1 hour.

In an embodiment, the extraction process intensified by Ultrasound-Assisted Extraction (UAE) consists of immersing a ground or unground biological material immersed in aqueous PG at 50-70° C. with stirring under ultrasound using an ultrasonic reactor operating at a frequency of 24 kHz and an ultrasonic power of 250 W The duration of the ultrasonic treatment was 1 hour. In another embodiment, the duration of the ultrasonic treatment was less than one hour, preferably about 30 minutes.

In an embodiment, the extraction process intensified by Microwave-Assisted Extraction (MAE) is performed on ground or unground raw material. Solid/liquid ratio, temperature and duration were about the same as conventional and ultrasonic treatments.

In a preferred embodiment, the extraction process is intensified by combination of ultrasound and Microwave involved successive UAE and MAE. This treatment consists of submitting unground or ground raw materials to ultrasound followed by MW, or conversely, starting with MW application and then submitting the whole to ultrasonic treatment. The duration of UAE and MAE is between 5-320 minutes, preferably between 10-120 minutes and most preferably 20-45 minutes, even more preferably 30 minutes.

In a preferred embodiment, liquid extracts obtained by maceration of plant materials in aqueous PG, according to the present invention, proved to be more performant in terms of global extraction yield and polyphenols content compared to extracts obtained by using water and 80% aqueous ethanol as extraction solvent. These results were obtained with different raw materials.

In a preferred embodiment, Iris rhizomes clearly show the superiority of the extracts obtained using aqueous PG (compared with more conventional solvents such as water and hydro-alcoholic mixture) over a wide range of biological activity such as antioxidant and antimicrobial activities. The present invention also demonstrates that extracts from rosemary leaves, verbena leaves, chamomile flowers and cistus aerial parts obtained with aqueous PG solutions presented comparable or even better antioxidant and antimicrobial activities compared to aqueous extracts. Thus, the present inventors further confirmed the real interest in using mixtures of 1,2-alkanediols and water as safe and more performant replacement to water or aqueous ethanol. Furthermore, obtained extracts are more stable compared to both aqueous and ethanol extracts obtained from the same biological material using the said extraction process.

Liquid extracts obtained according to the method described in the invention can be easily used for cosmetic formulation. These liquid extracts exhibit comparable or even better stability compared to aqueous and ethanol extracts from the same biological material.

In an embodiment, extracts obtained have a low bio-burden of less than 1000 colony forming units per gram, preferably less than 100 colony forming units per gram and most preferably less than 10 colony forming units per gram. The extracts are free of preservative agents that are known by the person skilled in the art to be capable of exerting a toxic effect in humans, particularly in terms of endocrine disruptors. The good microbial stability of these solvents provides a means of indirectly improving the safety of the extracts, particularly in view of the fact that it would appear contradictory to use a non-toxic solvent only to then proceed to add potentially toxic antimicrobial agents.

In an embodiment, the biological material can be a plant or part or parts thereof; an animal or part or parts thereof; algae, a lichen, a fungus, a yeast, a mold, or bacteria. For example, the part or parts of plants can be chosen from wood, roots, rhizome, bark, flowers, petals, sepals, seeds, fruits, aerial parts, in particular the stem and leaves. The plant material used can be differentiated or not differentiated.

In an embodiment, the method as disclosed in this invention is followed to extract rosemarinic acid from rosemary using pentylene glycol and water. In some embodiments, caprylyl glycol was additionally used to extract said compound. In a further embodiment, the extraction is enhanced by microwave and or ultrasound waves.

In an embodiment, the method comprises the steps of contacting the biological material with the extraction solvent, immersing the biological material in the extraction solvent, macerating or percolating or infusing the mixture, filtering and/or centrifuging the extraction product obtained, thereby obtaining a liquid extract, wherein said extraction solvent comprises water and 1,2-pentane glycol, preferably the extraction solvent further comprises caprylyl glycol.

In an embodiment, the method comprises the steps of contacting the biological material, preferably Rosmarin, Verbena, Cistus ladaniferus, Matricaria chamomilla or Iris germanica, with the extraction solvent, immersing the biological material in the extraction solvent, macerating or percolating or infusing the mixture, filtering and/or centrifuging the extraction product obtained, thereby obtaining a liquid extract, wherein said extraction solvent comprises water and 1,2-pentane glycol, preferably the extraction solvent comprises 20-40 wt. % 1,2-pentane glycol, more preferably the extraction solvent further comprises 2-10 wt. % caprylyl glycol.

In an embodiment, the method comprises the steps of contacting the biological material, preferably Cistus ladanifer, with the extraction solvent, immersing the biological material in the extraction solvent, macerating the mixture at 50-90° C. for 10-100 minutes, preferably at 60-80° C. and for 20-45 minutes, filtering and/or centrifuging the extraction product obtained, thereby obtaining a liquid extract, wherein said extraction solvent comprises water and 1,2-pentanediol, preferably the extraction solvent comprises 20-40 wt. % 1,2-pentanediol, more preferably the extraction solvent further comprises 2-10 wt. % caprylyl glycol.

The method is very suitable to extract polyphenolic compounds, especially Apigenin (50 μg/ml), ellagitannins (13 μg/ml) and kaempherol glucoside (5 μg/ml). Such high concentrations are difficult to obtain using conventional extraction methods. Furthermore, the extraction solvent used is desired in the obtained extraction product and can be utilized as such in cosmetic products.

In a second aspect, the invention relates to a cosmetic preparation comprising an extract obtainable by the method according to any of the previous embodiments, preferably containing the alkane diol. In a preferred embodiment of the invention, the cosmetic preparation was free of hexane, halogenated solvents, methanol and/or ethanol.

Using bio-based, safe compounds for extraction is preferred over using toxic conventional extraction agents such as hexane, methanol, ethanol, cyclohexane, butanol, chloroform, dichloromethane, etc. and results in an improved cosmetic preparation. No purification step is required to remove said toxic conventional extraction agents. Furthermore, no residues of said conventional extraction agents may be present in the end product for safety purposes. This differs for the extraction solvent used in this invention, as these compounds are even added to cosmetic or pharmaceutical preparations for its moisturising and anti-aging properties.

Cosmetic preparations are often used to fight skin aging effects. The aim of said cosmetic preparation is promoting the adhesion of the keratinocytes of the epidermal basal layer to the dermo-epidermal junction, in association with at least one collagen stimulating active agent for stimulating synthesis of collagen IV or of collagen VII.

This extract is used for the manufacture of a nutraceutical composition, of a dietetic or food product, of a nutritional supplement, of a pharmaceutical composition, in particular dermatological composition or of a cosmetic composition or of a nutraceutical, dietetic or food composition, nutritional, pharmaceutical, in particular dermatological or cosmetic comprising this extract.

In an embodiment, the extracted cosmetic and/or pharmaceutical compounds, preferably include phenolic acids and esters, flavonoids, secoiridoids, stilbenes and phenolic alcohols, antioxidants, vitamins, hormones, carotenoids, alkaloids, lipids, phenylpropanoids, flavor and taste modifiers, fragrances, biocides, antimicrobials, proteins, enzymes, colorings, pigments, surfactants, terpenoids including saponins, biological polymers, more preferably gallic acid, rosmarinic acid, hesperidin, curcumin, β-carotene and ferulic acid.

In a third aspect, the invention relates to a use of a solvent comprising water and one or a plurality of alkane diols with 5 through 12 carbon atoms to extract and/or dissolve biological compounds. In a particularly preferred embodiment, said extract is added in a cosmetic formulation, intended to be administered dermally and/or orally and/or for topical, rectal, nasal, auricular, vaginal and/or ocular application. In a further embodiment said extract is part of compositions with photo-protective, anti-photo-ageing, hypo-pigmenting, bleaching, anti-ageing, antioxidant, antiradical, metalloproteinase inhibiting, anti-inflammatory, skin soothing, and/or hydrating effect and/or where the composition reduces oxygen reactive species, activates collagen synthesis, restores the barrier function of the skin and/or improves the adhesion and/or cohesion of mammal cells.

Glycols are often used in moisturizers to enhance the appearance of skin by reducing flaking and restoring suppleness due to its function as a humectant. Other reported uses include skin-conditioning agent, viscosity-modifying agent, solvent, and fragrance ingredient. According to the present invention, diols and/or glycols will be added during extraction and are thus already present in the extract. This makes them more suitable and reduces the processing costs when converting said extracts into pharmaceuticals or cosmetic products.

In an embodiment, extracts obtained according to this invention, can be considered readily suitable as ingredients for cosmetics, personal care or pharmaceutical formulations. As these extracts comprise moisturizers and biological extracted compounds such as fragrances or pharmaceutical active compounds but no toxic, conventional extraction solvents, the extracts can be used as such, or after a limited purification step.

In an embodiment, the solvent comprising water and one or a plurality of C5-C12 alkane diols is used to extract and/or dissolve gallic acid, rosmarinic acid, hesperidin, curcumin, ferulic acid, proteins, lipids, preferably the solvent comprises PG and water. Said one or a plurality of C5-C12 alkane diols are suitable to extract high concentrations of desired compounds such as gallic acid, rosmarinic acid, hesperidin, curcumin, ferulic acid, proteins, lipids. Furthermore, alkane diols, especially 1,2 alkane diols are desired in cosmetic products for their skin improving properties.

Said alkane diols should thus not be removed from extraction products. In contrast to extraction methods using conventional solvents, an expensive purification is not required when producing cosmetic products using extraction methods described herein.

### EXAMPLES

Extraction procedures are described as well as analytical methods, which were used to characterize the raw materials and the extracts obtained. To evaluate the extraction efficiency of aqueous 1,2-alkanediols, exemplary studies were performed on different plant species (see Table 1). All investigated plant species are known for their interesting biological activities, especially related to their content of phenolic compounds.

TABLE 1
Natural raw materials used for extraction trials

| | | Initial moisture |
|---|---|---|
| Plant species | Plant part used | content (%) |
| Rosmarinus officinalis L. | Leaves | 8.7 ± 0.2 |
| Verbena officinalis L. | Leaves | 9.6 ± 0.4 |
| Cistus ladanifer | Aerial parts | 3.7 ± 0.2 |
| Matricaria chamomilla | Flowers | 9.2 ± 0.7 |
| Iris germanica | Rhizomes | 7.3 ± 0.2 |

All experiments were performed at ambient temperature. Each model substance was weighted into test tubes, and a precise volume of each solvent mixture was added in order to obtain saturated solutions. The test tubes were vortexed for 5 min and subsequently centrifuged (12000 rpm during 15 min at 25° C.). An aliquot of the supernatant was diluted for UV-spectrophotometric assay. Absorbance measurements were made at wavelengths adapted to each studied molecule. High Performance Liquid Chromatography (HPLC-DAD) was used for Rosmarinic Acid (RA) quantification. Determination of Radical Scavenging Capacity was conducted through DPPH assay.

Each experiment was performed in triplicate.

### (i) Conventional Maceration

Conventional solid/liquid extraction was performed during 60 min under stirring at a solid/liquid ratio of 1/10 (m/m). Briefly, fifty grams of studied plant materials were immersed into 500 mL of the selected solvent or solvent mixture in a double-jacket reactor. The extraction temperature was kept constant at 60±1° C. using a thermoregulation system connected to the double jacket of the reactor. Conventional extraction was followed by solid/liquid separation (filtration and centrifugation for 10 min at 8000 rpm).

### (ii) Ultrasound-Assisted Extraction (UAE)

Solid/liquid extraction under ultrasound was carried out at the same conditions as the conventional treatment (temperature, solid/liquid ratio, etc.). Ultrasound-Assisted Extraction (UAE) was performed in an ultrasonic reactor with a capacity of 1 L and an ultrasonic power of 250 W, operating at 25 kHz. The ultrasonic reactor consists of a stainless-steel vessel equipped with a double-layered mantle to allow temperature control with a thermoregulation system. For each experiment, the solid raw material was immersed into the solvent and submitted to ultrasound during 60 min. The solid matrix was homogenized into the solvent with a motorised stirrer. UAE was followed by solid/liquid separation (filtration and centrifugation at 8000 rpm).

To assess the grinding effect on the efficacy of extraction, UAE was also carried out in the case of ground raw materials. Two pre-treatments were carried out: (i) coarse grinding for obtaining particles size >400 μm and (ii) fine grinding to obtain particles <100 μm.

(iii) Microwave-Assisted Extraction

Microwave-Assisted Extraction (MAE) was carried out from ground or unground rosemary leaves immersed in the studied solvent at a solid/liquid ratio of 1/10. MAE was carried out in a microwave (MW) reactor during 60 min under stirring. MW power was limited to 1000 W and was varied to reach the set temperature and then to keep it constant during the extraction. MAE was followed by solid/liquid separation (filtration and centrifugation at 8000 rpm).

### (iv) Combined Ultrasound/Microwave Extraction

Successive ultrasound (US) and microwave (MW) treatments were carried out in order to assess the impact of combining both techniques: (i) USMW treatment, including 30 minutes of US application followed by 30 min extraction under MW and (ii) MWUS treatment, where MW was first applied for 30 minutes followed by 30 min of US treatment. US and MW treatments were each carried out as described in sections (ii) and (iii).

### Example 1. Aqueous Green PG for Solubilizing Natural Active Compounds

### Example 1.1. Aqueous PG for Dissolving Model Substances

The solubilisation power of aqueous Pentylene Glycol solutions (from 0 to 100% of PG) for five different model substances was compared to that of 80 wt. % aqueous ethanol. The experimental solubilities (w/v) in grams of the model substances per litre of the tested solvents are summarized in Table 2.

The results show that the ability and selectivity of aqueous diols for the extraction of certain components can be adjusted by the ratio of water and diol. The following observations were made for the different model substances:
Gallic acid (GA) is an antioxidant compound which occurs in a wide range of plant species. This compound is poorly soluble in water at 20° C. The solubility of GA in aqueous PG rises with increasing concentration of PG. It reaches a maximum at 70% PG, where the solubilisation power of water is exceeded by a factor of ˜12.4, while reaching 58.75% of the solubilization power of 80% ethanol. Lower concentrations of PG (30% and 50%) also show good solubilities for GA. At concentrations >70% of green PG, the experimental solubility of GA decreases significantly. For instance, the solubility of GA in 100% PG is only 32.3% compared to that of 70% PG. This result can be partly related to the viscosity increase with increasing concentration of PG.
Rosmarinic Acid (RA) is poorly soluble in water. The solubility increases significantly upon addition of PG until reaching a maximum solubility in 50% PG, equivalent to 92% of the solubility in 80% ethanol. Significant solubility of RA was also observed in 70% PG. The solubility decreased markedly at higher concentrations of PG.
Hesperidin (He) is a flavanone glycoside, predominantly found in lemon and orange. This bioactive compound is poorly soluble in water. Results showed that the solubility of hesperidin tends to increase with the proportion of bio-based PG in the aqueous solution until reaching a maximum solubility at 50% of PG, corresponding to 1.7 times the solubilization power of 80% aqueous ethanol. PG at 70% and 30% also showed great potential for He solubilization. It is worth mentioning that He solubility decreases significantly at higher proportions of PG (>70%).
It was found that the solubility of Curcumin (CU) followed a different trend compared to the previously studied molecules. The solubilization power of aqueous PG increases significantly with increasing concentration of PG. The solubility reaches a maximum in 90% PG, thereby exceeding the solubilization power of 80% ethanol. Note that PG at 100% has also a high solubilization power. It can be thus concluded that high proportions of PG are more suitable for dissolving curcumin.
The solubility of ferulic acid (FA) followed the same trend as that of CU. High concentrations of PG were found to be more suitable for the solubilization of FA. For example, the solubility of FA in 50% aqueous PG was 91.4 times higher than that in pure water. The values obtained compared well with those of 80% ethanol. The highest solubility was obtained in 70% PG. The solubility of FA in this aqueous mixture was 178.12 times higher than in water and 1.16 times higher than in 80% ethanol. Note that comparable solubilities were obtained in 80% and 90% PG. The case of FA thus confirmed once again the high potential of PG-based aqueous solutions, with high PG contents of 60 to 90% being preferred for lipophilic compounds.

TABLE 2
Solubility values (g of the model substance/litre of solvent) in reference solvents (100% H₂O and 80% EtOH) and
aqueous PG solutions. GA: Gallic acid; RA: Rosmarinic Acid; HE: Hesperidin; CU: Curcumin; FA: Ferulic Acid.

| Model | Solvent | | | | | | | |
|---|---|---|---|---|---|---|---|---|
| Substance | 100% H₂O | 10% PG | 30% PG | 50% PG | 70% PG | 90% PG | 100% PG | 80% EtOH |
| GA | 10.5 ± 1.1 | 22.5 ± 0.85 | 67.2 ± 4.8 | 95.1 ± 8.12 | 130 ± 5.79 | 74.3 ± 2.85 | 42 ± 0.54 | 221.3 ± 9.82 |
| RA | 6.6 ± 2.4 | 39.1 ± 2.2 | 200.2 ± 7.3 | 302.3 ± 9.4 | 167.1 ± 11.5 | 11.5 ± 1.2 | 12.8 ± 1.2 | 328.7 ± 7.6 |
| HE | 0.00 | 0.013 ± 0.003 | 0.177 ± 0.006 | 0.218 ± 0.024 | 0.19 ± 0.011 | 0.118 ± 0.012 | 0.0216 ± 0.002 | 0.13 ± 0.009 |
| CU | 3 × 10⁻⁴ ± | 0.01 ± 0.002 | 1.9 ± 0.045 | 3.65 ± 0.22 | 4.7 ± 0.84 | 6.53 ± 0.071 | 5.85 ± 0.31 | 5.46 ± 0.37 |
| | 1 × 10⁻⁴ | | | | | | | |
| FA | 0.55 ± 0.022 | 1.65 ± 0.1 | 19.82 ± 0.68 | 50 ± 1.5 | 97.4 ± 1.9 | 90 ± 2.8 | 61.7 ± 7.2 | 83.86 ± 2.76 |

### Example 1.2. COSMO-RS Calculation for Solubility Prediction

Experiments using the modelling software COSMO-RS (SCM BV/Netherlands, “Conductor like screening model for real solvents”) were performed with the aim of predicting the solvent mixture with the highest dissolution capacity for the desired molecules. For this purpose, the COSMO-RS software was used to predict the theoretical solubility of hesperidin (HE) and gallic acid (GA) in different binary mixtures of PG and water. The calculated values were then compared with the experimental results for these substances (see example 1.1).

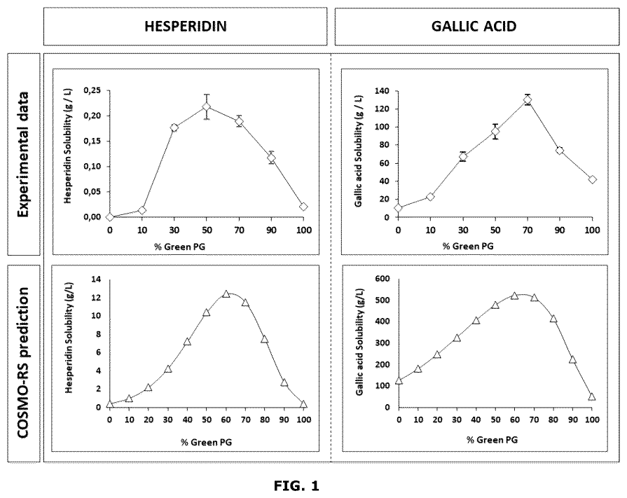

The results, shown in FIG. 1, indicate that the predictions of COSMO-RS correspond well with the experimental data. For both molecules, COSMO-RS showed the same solubility behaviour compared to experimental solubilities. Thus, a software like COSMO-RS can be considered as an efficient tool for the prediction of solubility properties. This can substantially speed up the selection of the right solvent for the desired purpose, thus allowing to limit the number of experiments to perform.

Similar simulations using COSMO-RS were performed for other target molecules with a wide range of polarities. Table 5 shows the calculated optimal concentrations of “green” PG in aqueous solutions that allow dissolution of the 11 active natural compounds. Table also provides examples of plant resources containing the above molecules, as well as potential cosmetic applications.

### Example 1.3. Bio-Based (PG:CG) Mixtures for Hesperidin Dissolving

Various mixtures of bio-based PG and bio-based caprylyl glycol (CG) were explored for dissolving hesperidin. The proportion of bio-based caprylyl glycol ranged from 0 to 30%. The experimental solubilities of Hesperidin depending on the proportion of caprylyl glycol are presented in Table 3.

According to our results, adding 1% of CG to PG increased the solubility of Hesperidin by a factor of 3.6 compared to pure PG. At 5% CG, the solubility was 5 times higher than in PG alone, thus reaching 82.4% of the solubilisation power of 80% ethanol.

The result presented in this example confirm that addition of CG can boost the solubilization power of green PG. It can be therefore concluded that mixtures of alkanediols can represent a green and efficient solution, in particular for dissolving water-insoluble or poorly soluble compounds such as hesperidin.

TABLE 3
Hesperidin solubility (g of He/litre of solvent) into reference
solvent (100% PG, and 80% EtOH) and (PG:CG) solutions

| | Model substance |
|---|---|
| Solvent | Hesperidin |
| 100% PG | 0.022 ± 0.002 |
| 1% CG | 0.078 ± 0.005 |
| 5% CG | 0.11 ± 0.006 |
| 10% CG | 0.071 ± 0.003 |
| 20% CG | 0.071 ± 0.007 |
| 30% CG | 0.06 ± 0.01 |
| 80% EtOH | 0.13 ± 0.009 |

### Example 2. PG/Water/CG Ternary System

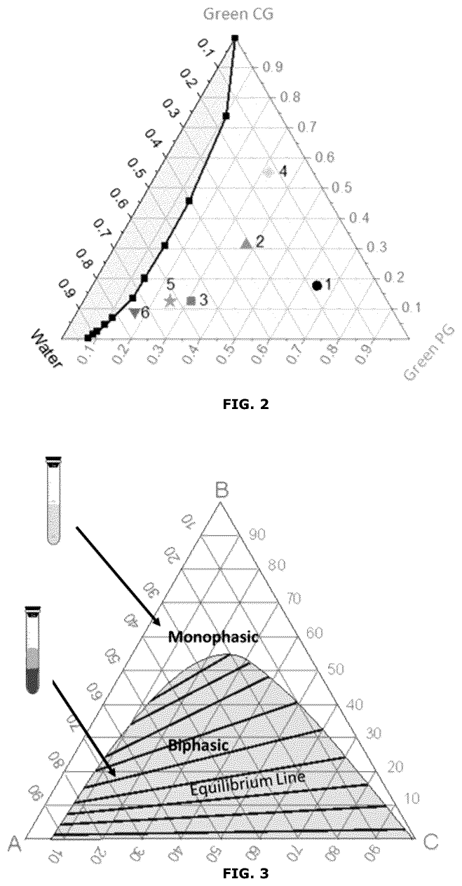

Regarding solubility in the ternary mixture of PG/CG/water, six different mixtures out of the monophasic region of the ternary system were prepared (see FIG. 2): Three water-rich solvent mixtures, one with a high percentage of PG, one with a high percentage of CG and one with a similar percentage of all three components. All the alkanediols used in this study were bio-based or “green”. The composition of the chosen solvent mixtures is summarized in table 4. All the solvent mixtures were tested according to the same experimental protocol for their dissolution capacity of rosmarinic acid, hesperidin, curcumin, ferulic acid and β-carotene.

TABLE 4
Composition of ternary PG/CG/water mixtures

| Mixture | Composition | | |
|---|---|---|---|
| No | % (PG) | % (CG) | % (Water) |
| 1 | 64.7 | 17.6 | 17.7 |
| 2 | 37.5 | 31.2 | 31.3 |
| 3 | 31.2 | 12.5 | 56.3 |
| 4 | 32.1 | 55.1 | 12.8 |
| 5 | 25 | 12.5 | 62.5 |
| 6 | 16.65 | 8.9 | 74.45 |

### Example 2.1. PG/Water/CG as Solvent for Active Ingredients

The results from solubility studies with 5 different natural substances in 6 different monophasic solvent mixtures of the ternary system PG/water/CG are summarized in Table 5 (PG=biobased Pentylene Glycol, CG=biobased Caprylyl Glycol, HE=Hesperidin, RA=Rosmarinic Acid, FA=Ferulic Acid, CU=Curcumin, β-C=b-carotene):
Hesperidin (HE) showed the highest solubility in solvent mixture No. 3, which consists of 56.3% water, 12.5% CG, and 31.2% PG. The observed concentration was 3.6 times higher than that in 80% ethanol, which is commonly considered an excellent reference solvent for hesperidin. Mixtures 2 and 5 also showed similarly good solubilizing properties for hesperidin (HE). It is noteworthy that point 5 represents a good compromise between physicochemical properties and production costs thanks to its high content of water (62.5%). All six ternary blends tested showed high solubility for He, which is completely insoluble in water.
Rosmarinic acid (RA) was most soluble in mixtures containing larger amounts of water (points 3, 5 and 6). For example, point 3 solubilized 26.8 times more RA compared to water. Point 5 showed a 27.2 times higher solubilisation power than water. Consequently, the results presented in this example clearly demonstrate the great interest in using a ternary system based on green PG as a solubilizer for poorly water-soluble molecules such as RA.
Ferulic Acid (FA) showed higher solubility in those mixtures containing lower amounts of water. The use of point 1 with 17.7% water, 64.7% PG, and 17.6% CG resulted in 117 times higher solubility of FA compared to water (64.34 g FA g/L for point 1 versus 0.55 g FA g/L in water). In addition, this mixture exhibits 76.7% of solubility compared to 80% ethanol. Other ternary mixtures tested also have great potential to dissolve FA. For example, FA a showed 26.5 times higher solubility in point 6 (74.45% water, 8.9% CG, and 16.65% PG) than in water. FA is usually dissolved in alkaline aqueous solutions in the form of its phenolate salt. Ternary systems containing water, bio-based PG and CG are a more skin-friendly alternative to this. Neutral or acidic solutions of polyphenols are also known to be more stable to air oxidation and have an overall lighter colour compared to alkaline phenolate solutions. Therefore, the solutions according to the invention are attractive for use as ingredients in personal care products where a light colour, long shelf life and good skin tolerance are required.

TABLE 5
Solubility values (g of model substance/litre of solvent) into 6 points within
the monophasic area of PG/water/CG system.

| | Composition of ternary | | | | | | | |
|---|---|---|---|---|---|---|---|---|
| | solvent mixture (%) | | | Concentration of natural substance in a saturated solution (g/L) | | | | |
| Nº | % PG | % CG | % H₂O | HE | RA | FA | CU | B-C |
| 1 | 64.7 | 17.6 | 17.7 | 0.34 ± 0.03 | 66.91 ± 4.46 | 64.34 ± 1.18 | 12.68 ± 0.46 | 0.86 ± 0.061 |
| 2 | 37.5 | 31.2 | 31.3 | 0.44 ± 0.03 | 118.47 ± 5.54 | 54.92 ± 0.88 | 9.98 ± 0.65 | 0.68 ± 0.065 |
| 3 | 31.2 | 12.5 | 56.3 | 0.46 ± 0.01 | 176.78 ± 8.20 | 30.72 ± 1.56 | 3.70 ± 0.18 | 0.48 ± 0.034 |
| 4 | 32.1 | 55.1 | 12.8 | 0.30 ± 0.03 | 68.41 ± 1.72 | 49.51 ± 0.48 | 11.70 ± 0.21 | 1.86 ± 0.047 |
| 5 | 25 | 12.5 | 62.5 | 0.40 ± 0.03 | 179.49 ± 16.76 | 25.78 ± 0.33 | 3.51 ± 0.30 | 0.34 ± 0.028 |
| 6 | 16.65 | 8.9 | 74.45 | 0.31 ± 0.01 | 139.55 ± 14.30 | 14.52 ± 0.88 | 2.46 ± 0.47 | 0.30 ± 0.052 |

The solubility of curcumin (CU) in water is very limited (0.5 g CU/L). Accordingly, the ternary mixtures with low water content were particularly suitable for solubilization of CU. The most efficient of the tested mixtures was point 1 (17.7% water, 17.6% CG, 64.7% PG). It provided a 1.6-fold higher solubility compared to 80% ethanol (12.7 g CU/L for point 1 versus 7.9 g CU/L in 80% ethanol). Points 2 and 4 were also identified as good candidates for CU solubilization. It is noteworthy that point 6, which contains more than 74% water, can be considered as an economical alternative to water since dissolves 3.5 g CU/L. This result is interesting in that curcumin is hardly soluble in water and therefore commonly dissolved in aprotic organic solvents such as acetone and ethyl acetate. Consequently, the present examples provide a green and safe alternative to irritating and harmful solvents while maintaining a high solubility of CU.
β-carotene is completely insoluble in water and protic organic solvents such as lower alcohols. The substance is however soluble in non-polar organic solvents such as hexane. It is therefore highly demanded to find green and safe alternatives able to solubilize this particularly hydrophobic molecule (log P=15.51±0.43). All ternary mixtures tested allowed a significant increase in the solubility of β-carotene compared to water. Point 4 (12.8% water, 55.1% CG and 32.1% PG) showed the highest solubilizing capacity (1.9 g β-C/L), followed by point 1 (0.85 g β-C/L). Interestingly, three times more β-C could be dissolved with point 4 than with hexane, which is generally considered the best solvent for this substance.

### Example 3. Polyphenols Extraction Through Conventional Maceration

### Example 3.1. Verbena Leaves

Dry Verbena leaves were subjected to conventional extraction by maceration. The extraction performance of aqueous PG in comparison with the reference solvents water and 80% ethanol was assessed in terms of global extraction yields (expressed as g dry weight of extract/100 g of leaves dry weight) and Total Phenolic Content (TPC, expressed as mg of Gallic acid equivalents [GAE]/g of extract). Table 6 summarizes the obtained results.

From these results, it can be concluded that 50% PG gave the comparatively highest extraction yield, followed by 30% PG. PG at 50% was also the most efficient solvent in terms of TPC. This solvent extracted 1.8 times more polyphenols compared to water. It also exceeded the extraction efficiency of 80% ethanol by 12.1%. 30% PG is also interesting for its ability to extract phenols, especially compared to aqueous extraction.

TABLE 6
Extraction yields and TPC from Verbena leaves using water, 30%
aqueous green PG, 50% aqueous green PG and 80% aqueous ethanol

Extraction performance

| | Global extraction | Total |
|---|---|---|
| | yield (g DW/100 g | phenolic content |
| Solvent | leaves DW) | (mg GAE/g extract) |
| Water | 11.3 ± 0.58 | 1.2 ± 0.10 |
| 30% PG | 11.7 ± 0.20 | 1.4 ± 0.05 |
| 50% PG | 13.9 ± 0.11 | 2.1 ± 0.13 |
| 80% Ethanol | 10.2 ± 0.65 | 1.9 ± 0.06 |

Hence, the results presented in this example demonstrate the great potential of aqueous green PG to reach high yields and to obtain extracts of high quality in terms of TPC and RA contents.

### Example 3.2. Rosemary Leaves

Dry rosemary leaves were subjected to conventional extraction by maceration. The extracts obtained were analysed for global extraction yield, total phenolic content (TPC) and rosmarinic acid content (RA). The results are summarized in Table 7.

TABLE 7
Extraction yield, TPC and RA content of rosemary leaf
extracts obtained by conventional maceration.

Extraction performance

| | Extraction yield | TPC | RA Content |
|---|---|---|---|
| | (g DW/100 g | (mg RAE/g | (mg/g |
| Solvent | leaves DW) | extract) | extract) |
| Water | 15.8 ± 0.30 | 4.9 ± 0.09 | 0.86 ± 0.01 |
| 30% PG | 20.6 ± 0.20 | 8.1 ± 0.19 | 1.08 ± 0.02 |
| 50% PG | 22.9 ± 0.31 | 6.8 ± 0.22 | 1.09 ± 0.02 |
| 80% Ethanol | 20.7 ± 0.66 | 5.4 ± 0.20 | 1.22 ± 0.04 |

In terms of global extraction yield, green PG at 50% was the most performant solvent followed by ethanol and green PG at 30%. The three tested solvents performed better than water (see Table 7, column 1). Using 50% PG enabled to reach a 1.4 times higher extraction yield than by aqueous extraction (22.9% for 50% PG versus 15.8% for water, respectively).

Regarding polyphenols extraction (Table 7, second column), results showed that both aqueous PG solutions (at 30% and 50%) were more efficient than both water and 80% ethanol %. The extraction power of 30% PG was 1.65 and 1.5 times higher than that of water and 80% ethanol, respectively. Therefore, results in terms of extraction yields and TPC confirmed the high potential of aqueous PG which proved to be a performant solvent for RA extraction from coarsely ground Rosemary leaves.

The extraction power of aqueous PG solutions was further confirmed by the yields obtained for the component Rosmarinic Acid (RA), as shown in Table 7, third column. Using 30% PG allowed to extract 1.27 times more RA compared to aqueous extraction. Furthermore, this solvent reached 89% of the extraction capacity of 80% ethanol.

Hence, the results presented in this example demonstrate the great potential of aqueous PG to reach high yields and to obtain extracts of high quality in terms of TPC and RA contents.

### Example 3.3. Cistus Aerial Parts

Solid/liquid extraction of coarsely ground dry aerial parts of Cistus was performed using water, 80% ethanol and aqueous PG solutions (30% and 50% of PG). The results are presented in Table 8.

TABLE 8
Extraction yields, TPC and RSC content from Cistus aerial parts

| | Extraction performance | | |
|---|---|---|---|
| | Extraction yield | TPC | RSC |
| | (g DW/100 g | (mg GAE/g | (mg TE/g |
| Solvent | leaves DW) | extract) | extract) |
| Water | 21.8 ± 0.92 | 5.7 ± 0.22 | 0.43 ± 0.005 |
| 30% PG | 18.2 ± 0.58 | 8.6 ± 0.42 | 0.40 ± 0.003 |
| 50% PG | 27.9 ± 0.16 | 7.9 ± 0.52 | 0.40 ± 0.008 |
| 80% Ethanol | 22.7 ± 1.09 | 3.4 ± 0.22 | 0.517 ± 0.006 |

In this study, 50% PG provided the highest extraction yield (1.23- and 1.28-times higher yields compared to 80% ethanol and water, respectively). The performance gain associated with aqueous PG was even more significant in terms of TPC (Table 8, second column). When 30% PG was used, 2.5 times more polyphenols were extracted compared to 80% ethanol. A similar improvement in performance was observed with 50% PG, which resulted in 2.3 times more polyphenols than 80% ethanol.

The radical scavenging capacities (RSC) of the obtained extracts, expressed as mg Trolox equivalent (TE)/g extract, are shown in the third column of Table 8. The results show that the extract obtained with 30% PG has 77.3% of the antioxidant capacity of the ethanolic extract. Using 50% PG, the corresponding extract reaches 76.6% of the antioxidant capacity of the ethanolic extract. Considering the obtained results, aqueous PG proved to be a good candidate for conventional extraction of Cistus aerial parts.

### Example 3.4. Chamomile Flowers

In the case of chamomile flowers, the four tested solvents were shown to provide similar yields under conventional maceration conditions (Table 9, first column).

TABLE 9
Extraction yields, TPC and RSC content from Chamomile flowers

Extraction performance

| | Extraction yield | TPC | RSC |
|---|---|---|---|
| | (g DW/100 g | (mg GAE/g | (mg TE/g |
| Solvent | leaves DW) | extract) | extract) |
| Water | 26.9 ± 1.27 | 0.8 ± 0.09 | 0.44 ± 0.003 |
| 30% PG | 24.9 ± 2.27 | 1.2 ± 0.04 | 0.45 ± 0.013 |
| 50% PG | 26.04 ± 1.22 | 1.5 ± 0.11 | 0.44 ± 0.014 |
| 80% Ethanol | 21.5 ± 1.09 | 1.3 ± 0.07 | 0.51 ± 0.027 |

In terms of polyphenols extraction, 30% PG and 50% PG performed similarly to 80% ethanol and significantly better than water (Table 9, second column). 30% PG provided extracts containing 1.5 times more polyphenols than aqueous extracts. The same positive contribution was noticed in the case of 50% PG which provided extracts with 1.83 times more polyphenols.

Both aqueous PG solutions allowed to obtain extracts with high antioxidant power. 30% PG was sufficient to obtain extracts with an antioxidant capacity 85.3% of that of ethanolic extracts. Using 50% PG allowed to reach 86.4% of the efficacy of 80% ethanol (see Table 9, third column).

According to results presented in this example, both aqueous PG solutions represent very promising candidates for extraction of chamomile flowers. Their use allowed to reach extraction performances comparable or superior to 80% ethanol.

### Example 3.5. Iris Rhizomes

Under maceration, 30% and 50% PG yielded extracts comparable to those obtained with 80% ethanol in terms of global extraction yield, TPC, and RSC (Table 10).

Both aqueous PG solutions allowed to significantly improve the extraction performance compared to water and 80% ethanol. For example, 30% PG allowed to obtain 4 times more polyphenols compared to aqueous extraction. The same performance gain was attributed to 50% PG. Both solvents provided comparable phenolic contents as the ethanolic extracts (Table 10, second column).

TABLE 10
Extraction yields, TPC and RSC content from Cistus aerial parts

Extraction performance

| | Extraction yield | TPC | RSC |
|---|---|---|---|
| | (g DW/100 g | (mg GAE/g | (mg TE/g |
| Solvent | leaves DW) | extract) | extract) |
| Water | 22.3 ± 1.73 | 0.4 ± 0.02 | 0.06 ± 0.003 |
| 30% PG | 21.5 ± 1.35 | 1.5 ± 0.01 | 0.20 ± 0.004 |
| 50% PG | 20.2 ± 0.91 | 1.5 ± 0.07 | 0.28 ± 0.020 |
| 80% Ethanol | 19.1 ± 0.79 | 1.4 ± 0.04 | 0.28 ± 0.003 |

Results related to RSC further proved the potential of aqueous PG to obtain extracts enriched with compounds of interest, in particular with antioxidants. Extracts obtained with 50% PG exhibit an antioxidant activity comparable to that of ethanolic extracts and considerably higher than that of aqueous extracts. Compared to water, this aqueous mixture provided extracts with an antioxidant capacity which is 4.6-fold higher than that of aqueous Iris extracts. Extraction with 30% PG resulted also in significantly higher antioxidant capacity compared to water. The corresponding RSC was 3.3 times that of aqueous extracts (Table 10, third column).

Hence, results presented in this example fully demonstrate the potential of aqueous PG mixtures for the extraction of natural compounds, and in particular polyphenolic antioxidants. Furthermore, the various results show the potential of obtained liquid extracts for cosmetic, food (human and animal), pharmaceutical or nutraceutical applications, in particular as antioxidant agents.

### Example 4. Ultrasound Assisted Extraction with Aqueous PG

### Example 4.1. Verbena Leaves

The results of Ultrasound Assisted Extraction (UAE) of Verbena leaves are presented in Table 11. The global extraction yield (first column) and TPC (second column) of the aqueous PG extracts were higher than in the extracts obtained with both water and 80% ethanol. 30% PG is particularly interesting since it provides an extraction yield 1.5 times that of ethanolic extraction (first column) with a performance gain of +9.5% in terms of polyphenols extraction (second column).

TABLE 11
UAE of Verbena leaves: Extraction yields and TPC

Extraction performance

| | Extraction yield | TPC |
|---|---|---|
| Solvent | (g DW/100 g leaves DW) | (mg GAE/g extract) |
| Water | 12.1 ± 1.11 | 1.33 ± 0.03 |
| 30% PG | 13.3 ± 0.19 | 2.08 ± 0.09 |
| 50% PG | 12.5 ± 0.55 | 1.91 ± 0.12 |
| 80% Ethanol | 9.0 ± 0.90 | 1.90 ± 0.19 |

### Example 4.2. From Rosemary Leaves

50% PG provided the highest global extraction yield in UAE of Rosemary leaves, as shown in Table 12. However, 30% PG appears as the most efficient solvent in terms of extract quality (TPC and RA content). Compared to 80% ethanol, this aqueous solution allowed to obtain extracts with 1.5 times more polyphenols and a +6.4% performance gain in terms of RA content. It is important to mention that both aqueous PG solutions are particularly more adapted to ultrasonic extraction from rosemary leaves compared to water.

TABLE 12
UAE of Rosemary leaves: Extraction yields and TPC

Extraction performance

| | Extraction yield | TPC | RA Content |
|---|---|---|---|
| | (g DW/100 g | (mg RAE/g | (mg/g |
| Solvent | leaves DW) | extract) | extract) |
| Water | 18.0 ± 0.57 | 5.7 ± 0.02 | 0.95 ± 0.014 |
| 30% PG | 17.4 ± 0.39 | 8.6 ± 0.26 | 1.51 ± 0.004 |
| 50% PG | 24.5 ± 0.64 | 7.2 ± 0.01 | 1.16 ± 0.042 |
| 80% Ethanol | 22.6 ± 0.04 | 5.6 ± 0.04 | 1.09 ± 0.012 |

Thus, it can be concluded from this example that 30% aqueous PG can be favourably selected for RA extraction under US. The obtained extracts exhibit a higher RA content compared to extraction with the other tested solvents.

### Example 4.3. Effect of Grinding on the Performance of Ultrasound-Assisted Extraction

This example aims at assessing the impact of grinding on the extraction performance. Solid/liquid extraction in aqueous PG was performed with the following materials: (i) UAE of unground Rosemary leaves, (ii) UAE of coarsely ground Rosemary leaves (particles size >400 μm) and (iii) UAE of finely ground Rosemary leaves (particles size <100 μm).

Extraction performances were then evaluated in terms of global extraction yield, TPC, and content of targeted active substance (Rosmarinic Acid, RA) in the final extract. 30% PG was used as solvent for all extractions.

As illustrated in Table 13, results proved the considerable impact of grinding.

TABLE 13
Impact of grinding on the global extraction yield,
TPC and RA content of Rosemary leaves extracts.

| | Extraction performance with 30% PG | | |
|---|---|---|---|
| | Extraction yield | TPC | RA Content |
| | (g DW/100 g | (mg RAE/g | (mg/g |
| Prior treatment | leaves DW) | extract) | extract) |
| Without grinding | 6.1 ± 0.5 | 2.4 ± 0.02 | 0.37 ± 0.017 |
| Coarse grinding | 17.4 ± 0.4 | 8.6 ± 0.26 | 1.51 ± 0.003 |
| Fine grinding | 20.7 ± 0.9 | 8.6 ± 0.15 | 1.42 ± 0.025 |

Combined with US, coarse grinding allowed to multiply the global extraction yield by a factor or 2.8 (6.1 g DW/100 g leaves DW for UAE from unground leaves and 17.4 g DW/100 g leaves DW from coarsely ground leaves, respectively). Fine grinding further increased the extraction yield to 20.7 g DW/100 g leaves DW (Table 13 column).

Differences between coarse and fine grinding were less apparent in terms of TPC (Table 13, second column). Combining US and coarse grinding further multiplied the TPC by 3.6. Unexpectedly, fine grinding seemed to not improve polyphenols extraction. Therefore, based on Folin-test, coarse grinding combined with US appears to be a particularly efficient process.

In a similar manner, ultrasonic treatment of coarsely ground leaves was shown to provide the highest RA content (Table 13, third column). Accordingly, a more efficient extraction was achieved by coarse grinding of rosemary leaves (>400 μm). This process made it possible to obtain extracts with higher polyphenolic content and higher RA content compared to conventional maceration.

### Example 5. Microwave-Assisted Extraction (MAE) of Polyphenols

### Example 5.1. Extraction Performance from Rosemary Leaves

Microwave-Assisted Extraction (MAE) was carried out with coarsely ground rosemary leaves using: (i) 30% PG; (ii) 50% PG; (iii) water and (iv) 80% ethanol. Results in terms of global extraction yield, total phenolic content (TPC) and RA content are presented in Table 14.

TABLE 14
MAE of Rosemary leaves: Extraction yields, TPC and RA content

Extraction performance

| | Extraction yield | TPC | RA Content |
|---|---|---|---|
| | (g DW/100 g | (mg RAE/g | (mg/g |
| Solvent | leaves DW) | extract) | extract) |
| Water | 17.9 ± 0.56 | 5.5 ± 0.15 | 0.75 ± 0.011 |
| 30% PG | 20.7 ± 0.44 | 7.7 ± 0.21 | 1.16 ± 0.06 |
| 50% PG | 21.5 ± 1.63 | 7.0 ± 0.08 | 1.07 ± 0.13 |
| 80% Ethanol | 20.0 ± 0.13 | 6.4 ± 0.18 | 1.17 ± 0.019 |

In terms of total dry matter content, both aqueous PG solutions performed similarly to 80% ethanol and significantly better than water.

A similar conclusion can be made on the basis of polyphenols content. Both PG mixtures allowed to enhance phenolic content compared to reference solvents (water and 80% ethanol). For example, +20.3% polyphenols were obtained with 30% PG compared to ethanolic extraction. Compared to water, using 30% PG provided a performance gain of +40% in terms of TPC.

Further, 30% PG showed a high selectivity for RA. Its extractant power for RA is comparable to that of 80% ethanol, commonly known as reference solvent for RA. Moreover, the use of 30% PG made it possible to extract 51.2% more RA compared to aqueous extraction.

It can be therefore concluded that aqueous PG combined with MW allows to obtain extracts enriched with targeted compounds. These bio-based solvents were shown to be as efficient as 80% ethanol while being as safe and as eco-friendly as water. Meeting both greenness and efficiency requirements represents the main achievement of the developed process using solvents according to the invention.

### Example 5.2. Effect of Grinding on the Performance of MAE

MAE was performed with 30% PG as solvent for the extraction of (i) unground leaves, (ii) coarsely ground leaves (particles size >400 μm) and (iii) finely ground leaves (particles size <100 μm) of Rosemary. The contribution of prior grinding was assessed in terms of global extraction yield, TPC and RA content. Results revealed a significant impact related to size reduction (Table 15).

TABLE 15
MAE: impact of prior treatment on the global extraction
yield, TPC and RA content of Rosemary leaf extracts

| | Extraction performance | | |
|---|---|---|---|
| | Extraction yield | TPC | RA Content |
| | (g DW/100 g | (mg RAE/g | (mg/g |
| Prior treatment | leaves DW) | extract) | extract) |
| Without grinding | 5.5 ± 0.5 | 2.1 ± 0.20 | 0.24 ± 0.004 |
| Coarse grinding | 20.7 ± 0.4 | 7.7 ± 0.21 | 1.15 ± 0.058 |
| Fine grinding | 26.0 ± 0.7 | 8.7 ± 0.26 | 0.91 ± 0.013 |

Combining 30% PG with fine grinding allowed to obtain a global extraction yield 4.7-fold higher than that obtained from unground leaves. Coarse grinding also improved the extraction yield which was multiplied by 3.7 compared to MAE from unground leaves (Table 15, first column).

In a similar manner, a higher grinding level resulted in significantly increased phenolic contents. Coarse grinding made it possible to increase the polyphenols content by a factor of 3.7. Further size reduction further increased the TPC. Extraction from finely ground leaves allowed to obtain polyphenol concentrations 4.1 and 1.13 times higher compared to extraction from unground and coarsely ground leaves, respectively (Table 15, second column).

The positive contribution of coarse grinding was confirmed in terms of RA content in the extracts (Table 15, third column). This grinding level was shown to provide the highest RA content (4.5 times higher compared to MAE from unground leaves). The fine grinding seemed to not improve overall extracts quality. Due to the higher global yields reached with this higher grinding level, the obtained extracts contain additional, potentially undesired compounds. This means that coarse grinding provided a higher selectivity towards higher contents of the targeted antioxidant compounds.

In conclusion, coarse grinding was found to be the most appropriate prior treatment for MAE of rosemary leaves.

### Example 6. Impact of Combined Us/Mw on Polyphenols Extraction from Rosemary Leaves

In this part, conventional maceration as well as the two individual intensified extraction techniques (UAE and MAE) are compared to combinations of the two techniques: (i) USMW consisting of treating raw material by US followed by 30 minutes under MW and (ii) MWUS, which includes MW treatment for 30 minutes and then US treatment for the same duration.

These different techniques are compared in terms of their extraction performance (Extraction yield, TPC and RA content). Results are shown in Table 16.

TABLE 16
Extraction performance from rosemary leaves:
individual techniques (maceration, UAE, MAE)
compared to combined techniques (USMW, MWUS)

| | Extraction performance | | |
|---|---|---|---|
| | Extraction yield | TPC | RA Content |
| | (g DW/100 g | (mg RAE/g | (mg/g |
| Solvent | leaves DW) | extract) | extract) |
| Maceration | 20.6 ± 0.19 | 8.1 ± 0.19 | 1.08 ± 0.02 |
| UAE | 17.4 ± 0.39 | 8.6 ± 0.26 | 1.51 ± 0.003 |
| MAE | 20.7 ± 0.44 | 7.7 ± 0.20 | 1.16 ± 0.06 |
| USMW | 20.5 ± 0.82 | 7.5 ± 0.11 | 1.19 ± 0.005 |
| MWUS | 19.6 ± 2.45 | 7.4 ± 0.23 | 1.14 ± 0.04 |

Using 30% PG, similar global extraction yields, comparable yields were reached for all the tested individual and combined extraction technique (Table 16, first column). The same trend was observed in terms of polyphenols extraction (Table 16, second column) and RA contents (Table 16, third column). In all investigated cases, individual and combined intensified extraction techniques provided extracts of similar quality.

Based on the results presented in this example, it can be concluded that the two tested combinations had no significant impact compared to the individual techniques. However, it has been clearly demonstrated that aqueous PG is compatible with a wide range of extraction techniques including conventional (maceration) and intensified techniques (UAE and MAE), as well as successive combinations thereof (USMW and MWUS).

### Example 7. Aqueous PG as Solubilizer for Lipophilic Ingredients Preparation of Ternary Systems

The ternary systems studied consisted of water and an organic solvent that is immiscible with water. A third liquid was added that is soluble in each of the other two components. This third liquid acts as a hydrotrope, which increases the solubility of the two immiscible liquids in each other. Corresponding phase diagrams were created, such as the example shown in FIG. 3. The white areas indicate a single-phase mixture (clear solution), while the grey areas show a biphasic mixture. Understanding the behaviour of these mixtures allows for the creation of stable ternary solvent systems.

### (i) Ternary Systems Using Pure Solvents

Different ternary systems were tested to investigate the possibility of formulating stable single-phase mixtures.

For the mixture of bio-based Pentylene Glycol (PG), water and 2-methyloxolane (2-MeOx), binary mixtures of water and PG (0:100, 10:90, 20:80, 30:70, 40:60, 50:50, 60:40, 70:30, 80:20, 90:10, 100:0) were prepared in test tubes. Then 2-MeOx was added dropwise (10 μL) as the third component until the phase transition could be observed with the naked eye (transition of the liquid from clear to turbid). In an analogous manner, the binary mixture of PG and 2-MeOx was prepared, and water was added as the third component. After observing the phase transition, the total weight of the mixture was determined, and the composition was recorded in the ternary phase diagram. All sample solutions were prepared by weighing and the measurements were performed at room temperature.

The same procedure was used to establish the ternary diagrams of PG/hexane/water, PG/ethanol/water, and PG/bio-based Caprylyl Glycol (CG)/water. The tests with the PG/CG/water system were carried out at 35° C. since CG has a melting point of 30° C. and the phase behaviour was easier to observe in this way.

### (ii) Ternary Systems Including Oxygenated and Non-Oxygenated Monoterpenes

Binary mixtures of water and PG (0:100, 10:90, 20:80, 30:70, 40:60, 50:50, 60:40, 70:30, 80:20, 90:10, 100:0) were prepared in test tubes. Then the selected monoterpenes (carvacrol, carvone, geraniol, limonene, α-pinene, α-terpinene) were added dropwise (10 μL) to the binary mixtures and mixed. The addition of the monoterpenes was continued until the clear liquid changed to a turbid liquid. The test tubes were then shaken for 2 minutes and allowed to settle for 24 hours to check whether or not the biphasic liquid changed to a monophasic liquid. In the case of thymol, very small amounts of the solid sample were added to the binary mixtures of water/PG until a turbid liquid was visible to the naked eye.

(iii) Ternary Systems Including Essential Oils

Essential oils (thyme, citronella, eucalyptus and lavender) were added dropwise to the binary mixtures of water/PG until a turbid liquid was observed. After the addition of each drop (10 μL) and mixing, the samples were rested for 2 minutes to see whether there is a phase separation or not.

### (iv) Ternary Systems Including Vegetable Oils

In an analogous procedure, vegetable oils (pomegranate seed, plum kernel and hemp seed) were used to formulate ternary systems with water and PG.

### Example 7.1. Aqueous PG for Stabilizing Monoterpenes/Essential Oils

Ternary systems were formulated with water, PG and oxygenated or non-oxygenated monoterpenes.

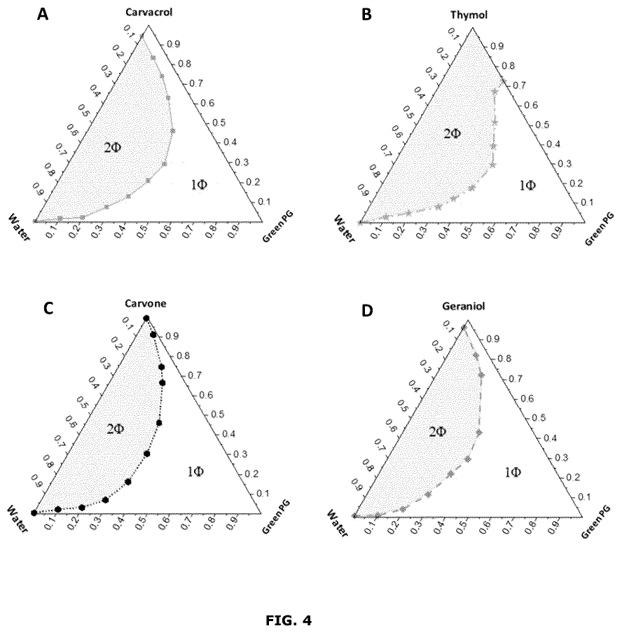

FIG. 4 shows the obtained phase diagrams for ternary systems formed with aqueous PG and the four oxygenated monoterpenes carvacrol, thymol, carvone and geraniol.

Results showed similar monophasic areas for the 4 tested monoterpenes. It turned out that aqueous PG, at defined proportions, can be successfully used for dissolving carvacrol, thymol, carvone and geraniol. For example, self-stabilized monophasic solutions are formed with 2.5% of water, 7.5% of PG and 90% of carvacrol. Similar water and PG proportions can be used to obtain stable solutions of thymol, carvone or geraniol (FIG. 4).

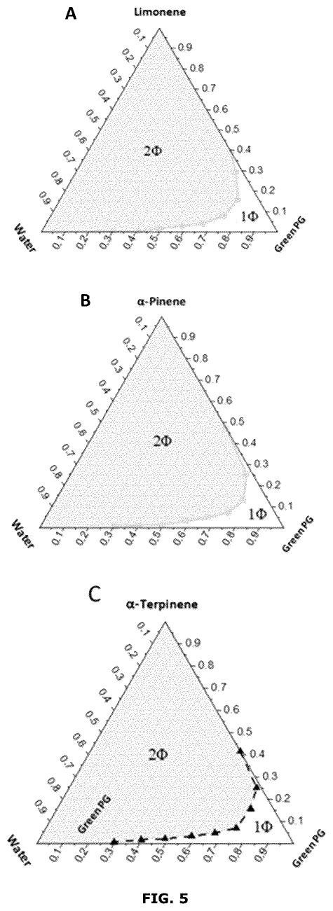

Limonene, α-pinene and α-terpinene were selected as examples of non-oxygenated monoterpenes. Their ternary systems with water and green PG are shown in FIG. 5.

It is apparent from these diagrams that monophasic regions are considerably reduced compared to oxygenated monoterpenes. In general, non-oxygenated monoterpenes proportions should not exceed 40% of the final solution in order to form monophasic stable systems. Moreover, for the 3 studied compounds, high proportions of PG are required in order to formulate stable monophasic solutions. In the first instance (A), self-stabilized ternary mixture can be obtained with 85% PG, 10% water and 5% limonene. This result could be related to its highly hydrophobic character of the non-oxygenated terpenes. The same behaviour was observed in the case of α-pinene and α-terpinene.

Observations related to our studied non-oxygenated and oxygenated monoterpenes can be generalized to similar compounds. That way, results presented in this example could be considered as a decision tool for further applications of aqueous PG as cosmetic stabilizer.

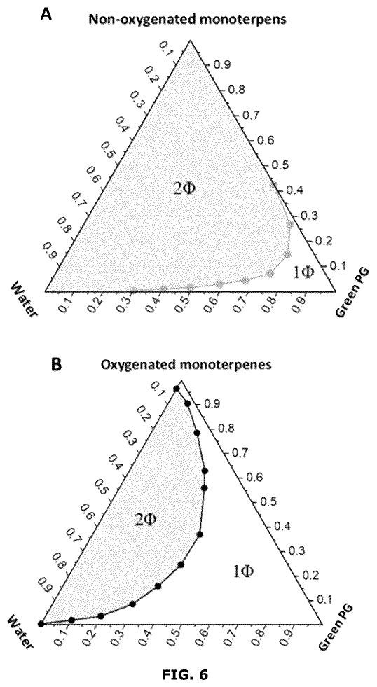

General behaviours of non-oxygenated and oxygenated monoterpenes, in mixture with water and green PG, are illustrated in FIG. 6.

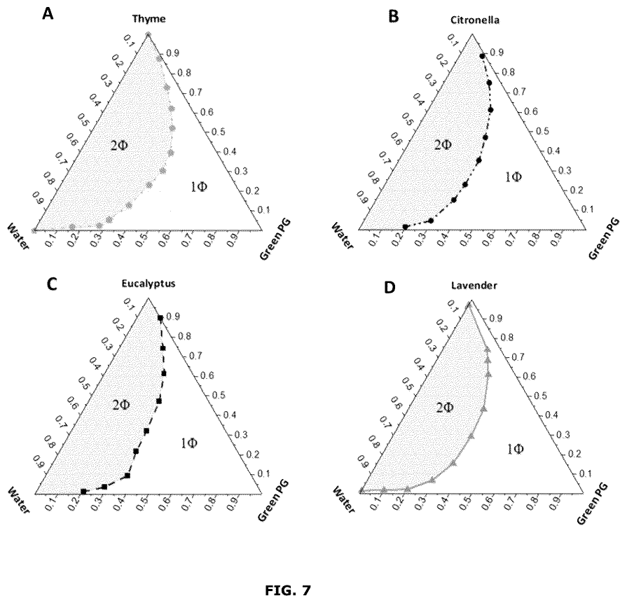

Ternary systems were also formulated at room temperature with water and PG in mixture with essential oils of (A) thyme, (B) citronella, (C) eucalyptus and (D) lavender. The corresponding phase diagrams are presented in FIG. 7.

Results showed that for the four tested essential oils monophasic regions are bigger than the biphasic areas. Thanks to those large monophasic areas, it is possible to choose the optimum compositions with targeted essential oil proportion for stability and formulation purposes. Furthermore, exceptionally high proportions of these essential oils can be used to obtain stable monophasic solutions. For example, a stable monophasic solution can be formulated with 2.5% of water, 17.5% of green PG and 90% of lavender essential oil.

Consequently, results presented in this example further proved the potential of aqueous PG, at defined proportions, as cosmetic stabilizer for essential oils or cosmetic formulation including essential oils.

It should be also pointed out that optimum compositions could be deduced from results related to major compounds of each oil, especially non-oxygenated and oxygenated monoterpenes. That way, the behaviour of thyme essential oil could be predicted from that of its two major compounds, namely thymol and carvacrol (compare FIG. 7.A to FIGS. 4.A and 4.B).

Thus, results presented in this example could be considered as very useful decision tools for formulators of cosmetics or perfuming products.

### Example 7.2. Aqueous PG for Stabilizing Vegetable Oil

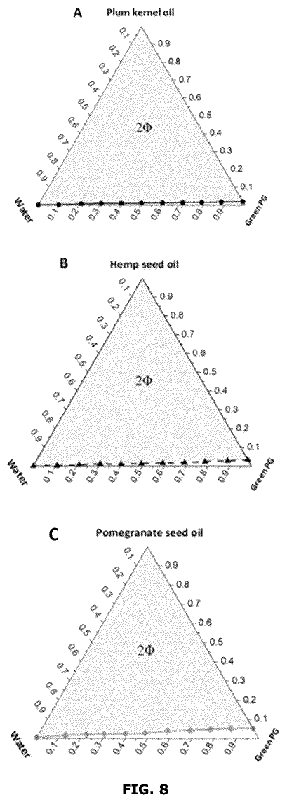

Ternary systems formulated at room temperature using water, PG, and vegetable oils (plum kernel, hemp seed and pomegranate seed oils) are shown in FIG. 8.

As depicted in FIG. 8, there is a small monophasic region with moderate oil content (no more than 0.06%) in the three studied PG/Water/vegetable oil systems. As possible applications, this type of ternary system could help to stabilize vegetable oils containing residual moisture, while meeting requirements for naturality and safety of the stabilizer.

### Example 7.3. PG-Based Ternary System as Decision Tool for Cosmetic Formulation

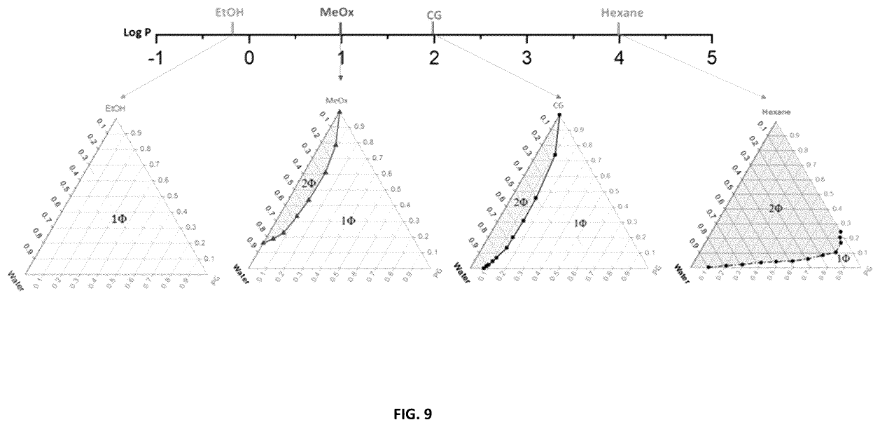

In this example, different PG-based ternary systems with water as second component were evaluated. For the third phase, pure solvents with different polarities were selected: ethanol, 2-methyloxolane (2-MeOx), Caprylyl Glycol (CG) and hexane. The obtained ternary systems are presented in FIG. 9.

The largest monophasic area was observed in the case of PG/water/EtOH since these three solvents are completely miscible with each other. On the other hand, the monophasic region was extremely reduced when using apolar solvents, especially in the case of hexane. Note that it was possible to obtain wide stable monophasic regions in the case of 2-MeOx (2-methyl oxolane=2-methyl tetrahydrofuran) and CG (Caprylyl Glycol).

The results presented in this example are of interest inasmuch as aqueous PG can be used as solubilizer, solvent or physical stabilizing additive in various types of cosmetic products, including oil-in-water and water-in-oil emulsions. This opens up new pathways for the utilisation of aqueous and preferably bio-based diols such as PG and CG.

### Example 8. Extract of Cistus ladanifer

### Preparation of Extraction Solvent

The extraction solvent was obtained with aqueous 30% 1,2-pentanediol as extraction solvent.

Preparation of Cistus ladanifer Extract

A mixture of 1:10 Cistus ladanifer/solvent was macerated at 60° C. for 60 min and subsequently filtered using a filter with <1 μm pore size.

### Analysis and Results

HPLC/MS analysis showed that significant amounts of polyphenolic compounds were extracted. Ellagitannins (13 mg/mL), gallic acid (120 μg/mL), apigenin (50 μg/ml), and kaempherol glucoside (8 μg/ml) were identified as major constituents. These substances are known for their antioxidant, antimicrobial, anti-inflammatory, UV-protective and skin-lightening effects.

The extract was applied as a 1% aqueous solution to reconstructed human epidermis two times within 48 hours and upregulation of several protein families was detected by HPLC/MS analysis. These changes in protein expression are linked to the following skin care benefits:
Antibacterial protection, NRLP inflammasome plays an important role in surveillance and defense against bacterial infections, +20% expression
Wound healing, proteins involved in responses to wounding, +10% expression
Aging, Cholesterol and Sphingolipids are key lipids in epidermal barrier function and facial skin moisture, +47% and +14% expression
Aging, Autophagy is an important salvage process for detoxification in aged skin, along with other proteins involved in redox balance and detoxification processes, +15-20% expression
Decrease in abundance of a sub-set of type II (acidic) keratins, −30%-40% decrease of expression.

1. Method for extracting biological material for obtaining cosmetic and/or pharmaceutical components in the obtained extract using an extraction solvent, comprising the steps of contacting the biological material with the extraction solvent, immersing the biological material in the extraction solvent, macerating or percolating or infusing the mixture, filtering and/or centrifuging the extraction product obtained, thereby obtaining a liquid extract, wherein said extraction solvent comprises water and one or a plurality of C5-C12 alkanediols.

2. Method according to claim 1, wherein said extraction solvent comprises 10-75 wt. % pentylene glycol, preferably said extraction solvent comprises 25-70 wt. % pentylene glycol.

3. Method according to any of claims 1-2, wherein said extraction solvent essentially consists of:
one or a plurality of C5-C12 alkanediols, wherein the total amount of C5-C12 alkanediols is 10-70 wt. % of the extraction solvent, preferably 25-70 wt. % of the extraction solvent; and
water, for the remainder of the extraction solvent.

4. Method according to any of claims 1-3, wherein said extraction solvent essentially consists of:
pentylene glycol, in an amount of 10-70 wt. % of the extraction solvent, preferably in an amount of 25-70 wt. % of the extraction solvent; and
water, for the remainder of the extraction solvent.

5. Method according to any of claims 1-4, wherein said extraction solvent comprises, preferably said extraction solvent essentially consists, of:
a first C5-C12 alkanediol, preferably said first C5-C12 alkanediol is pentylene glycol;
a second C5-C12 alkanediol, preferably said second C5-C12 alkanediol is caprylyl glycol; and
water.

6. Method according to any of the claims 1-5, wherein, the extraction solvent and the biological material are used with a weight ratio of between 1:1 and 100:1, preferably between 2:1 and 100:1, more preferably between 5:1 and 20:1, most preferably at a ratio of 10:1.

7. Method according to any of the claims 1-6, wherein the biological material prior to extraction is submitted to a shredding and/or a grinding step, preferably to an average particle size (D50) below 1 cm and more preferably below 1 mm.

8. Method according to any of the claims 1-7, wherein the biological material and/or the extraction solvent are heated prior or during the extraction, preferably to 30-100° C. and preferably to 50-70° C.

9. Method according to any of the claims 1-8, wherein the extraction is ultrasound-assisted preferably with a frequency between 10-40 kHz and more preferably 20-25 kHz and/or microwave-assisted with a frequency between 300 MHz and 300 GHz.

10. Method according to any of the claims 1-9, wherein said C5-C12 alkanediols are produced from renewable resources, preferably said C5-C12 alkanediols are biobased.

11. Method according to any of claims 1-10, wherein the plant biological material is selected from: cherry blossom, horsetail, plantain, saffron flowers, verbena leaves, rose of Jerico, rosemary, algae, fungi, Selaginella pulvinata, Tillandsia usnoides, citrus, Matricaria, iris, olive or other Embryophyta; the animal material is preferably selected from invertebrates; and prokaryotic biological material is preferably selected from gram-positive bacteria.

12. A cosmetic and/or pharmaceutical preparation comprising an extract obtainable by the method according to any of the claims 1-11, preferably containing the alkane diol.

13. Cosmetic and/or pharmaceutical preparation according to claim 12, free of hexane, halogenated solvents, methanol and/or ethanol.

14. Cosmetic and/or pharmaceutical preparation according to any of the claims 12-13, comprising cosmetic and/or pharmaceutical compounds, preferably said compounds including phenolic acids and esters, flavonoids, secoiridoids, stilbenes and phenolic alcohols, antioxidants, vitamins, hormones, carotenoids, alkaloids, lipids, phenylpropanoids, flavor and taste modifiers, fragrances, biocides, antimicrobials, proteins, enzymes, colorings, pigments, surfactants, terpenoids including saponins, biological polymers, more preferably gallic acid, rosmarinic acid, hesperidin, curcumin, β-carotene and ferulic acid.

15. Use of a solvent comprising water and one or a plurality of alkane diols with 5 through 12 carbon atoms to extract and/or dissolve biological compounds.
